(19) United States
(12) Patent Application Publication
(10) Pub. No.: US 2010/0184774 A1
(43) Pub. Date: Jul. 22, 2010

(54) METHODS OF TREATMENT

(76) Inventors: Kevin J. Duffy, Collegeville, PA (US); Connie Lynn Erickson-Miller, Collegeville, PA (US); Hideo Kikkawa, Tokyo (JP); Anna Coco Maroney, King of Prussia, PA (US)

Correspondence Address: GlaxoSmithKline;GLOBAL PATENTS -US, UW2220, P. O. BOX 1539, KING OF PRUSSIA, PA, 19406-0939, US

(21) Appl. No.: 12/602,623

(22) PCT Filed: May 29, 2008
(86) PCT No.: PCT/US08/65038

§ 371 Date: Dec. 1, 2009

Related U.S. Application Data
(60) Provisional application No. 60/941,330, filed on Jun. 1, 2007.

(51) Int. Cl. A61K 31/498; A61P 25/28; A61P 25/00; A61P 35/00; A61P 19/08; A61P 19/10; A61P 13/12; A61P 3/10; A61P 25/24; A61P 25/30; A61P 3/04; A61P 15/00

(52) U.S. Cl. 514/249

(57) ABSTRACT

This invention relates to a method of treating a disease state selected from: Alzheimer's disease, Down's syndrome, mental retardation, memory defects, memory loss, pancreatic cancer, bone resorption disease, osteoporosis, sickle cell anemia, chronic kidney disease, diabetes, depression, and subsets of depression including: alcoholism, anxiety, obsessive compulsive disorder, panic disorder, chronic pain, obesity, senile dementia, migraine, bulimia, anorexia, social phobia, pre-menstrual syndrome (PMS), adolescent depression, trichotillomania, dysthymia and substance abuse, in a mammal in need thereof, including a human, which comprises administering to such mammal a therapeutically effective amount of a selected substituted thiazol compound. The invention also relates to a method of enhancing cognition in a mammal in need thereof which comprises administering to such mammal a therapeutically effective amount of a selected substituted thiazol compound.

### FIELD OF THE INVENTION

This invention relates to a method of treating selected disease states in a mammal, including a human, by administration of a selected substituted thiazol compound and pharmaceutical compositions containing the same.

### BACKGROUND OF THE INVENTION

DYRK (dual-specificity tyrosine-phosphorylation-regulated kinase)/MNB (minibrain)/YAK kinases represent a family of dual-specificity kinases that autophosphorylate on tyrosine, serine and threonine, but appear to phosphorylate exogenous substrates only on serine or threonine residues [Lochhead et al., Biochem. J. (2003) 374, 381-391]. DYRK family members have been identified in all eukaryotes examined to date. Work in Saccharomyces cerevisiae, Drosophila and mice implicate this family of kinases in such fundamental processes as the regulation of cell proliferation, cytokinesis, developmental responses (such as the transition from a growth to differentiation phase) and, in higher eukaryotes, proper brain development. Distantly related to MAPK (mitogen-activated protein kinase) and Cdk (cyclin-dependent protein kinase) [Becker, et al., Prog. Nucleic Acid Res. Mol. Biol., 1999, Vol. 62, pp. 1-17., Miyata, et al., Biochem. Biophys. Res. Commun., 1999, Vol. 266, pp. 291-295, Widmann, et al., Physiol. Rev., 1999, Vol. 79, pp. 143-180, and Himpel, et al., J. Biol. Chem., 2000, Vol. 275, pp. 2431-2438.], the DYRK family is characterized by the presence of several distinct amino-acid sequences in the kinase domain, including an SSC motif following subdomain VII, conserved sequences HCDLKPEN and YXYIQSRFYR(S/A)PE in subdomains VI and VIII respectively, and a YXY motif in the kinase-domain-activation loop between subdomains VII and VIII, and by a DYRK homology (DH) box immediately preceding the kinase domain [Becker, et al., J. Biol. Chem., 1998, Vol. 273, pp. 25893-25902]. All DYRK proteins appear to have extended N- and/or C-terminal regions that display little homology to other proteins except closely related family members.

Several recent International Patent Publications refer to selected substituted thiazol compounds as inhibitors of hYAK3 (otherwise reported as, and hereinafter referred to as DYRK3) as having utility in treating diseases of the erythroid and hematopoietic systems, particularly anemias.

Other related DYRK family members include DYRK1a, DYRK1b, DYRK2 (otherwise reported as hYAK1), and DYRK4 (otherwise reported as hYAK2).

Inhibition of DYRK1a is described in Kim et al. Bioorg. Med. Chem. Lett. 16 (2006) 3772-3776, Woods et al. Biochem. J. (2001) 355, 609-615 (printed in GB), Ahn et al. Neurobiology of Disease 22 (2006) 463-472 and Altafaj et al. Human Molecular Genetics, 2001, Vol 10, No. 18, 1915-1923. Compounds that inhibit DYRK1a are considered useful in the treatment of Alzheimer's disease, Down's syndrome, mental retardation, memory defects, and memory loss. Compounds that inhibit DYRK1a are also considered to have utility in treating diabetes.

Inhibition of DYRK1b is described in Deng et al. Cancer Res 2006; 66(8) 4149-4158. Compounds that inhibit DYRK1b are considered useful in the treatment of pancreatic cancer.

Compounds that inhibit DYRK2 are considered useful in the treatment of bone resorption disease and osteoporosis.

Compounds that inhibit DYRK3 are considered useful in the treatment of sickle cell anemia and chronic kidney disease.

The present invention concerns selected substituted thiazol compounds that are know to inhibit DYRK3, and their novel use in the treatment of selected disease states.

### SUMMARY OF THE INVENTION

This invention relates to a method of treating selected disease states in a mammal, including a human, in need thereof, which comprises administration of a therapeutically effective amount of a selected substituted thiazol compound.

This invention relates to a method of treating depression, and subsets of depression including: alcoholism, anxiety, obsessive compulsive disorder, panic disorder, chronic pain, obesity, senile dementia, migraine, bulimia, anorexia, social phobia, pre-menstrual syndrome (PMS), adolescent depression, trichotillomania, dysthymia and substance abuse, which comprises administering a therapeutically effective amount of an inhibitor of DYRK1a, suitably the inhibitor of DYRK1a is a chemical compound.

This invention relates to a method of enhancing cognition, which comprises administering a therapeutically effective amount of an inhibitor of DYRK1a, suitably the inhibitor of DYRK1a is a chemical compound.

Included in the present invention are pharmaceutical compositions comprising a pharmaceutical carrier and compounds useful in the methods of the invention.

Also included in the present invention are methods of co-administering the selected substituted thiazol compounds with further active ingredients.

### DETAILED DESCRIPTION OF THE INVENTION

This invention relates to a method of treating selected disease states in a mammal, including a human, by administration of a selected substituted thiazol compound and pharmaceutical compositions containing the same.

The selected substituted thiazol compounds of the present invention treat the selected disease states of Alzheimer's disease, Down's syndrome, mental retardation, memory defects, memory loss, diabetes, depression, and subsets of depression including: alcoholism, anxiety, obsessive compulsive disorder, panic disorder, chronic pain, obesity, senile dementia, migraine, bulimia, anorexia, social phobia, pre-menstrual syndrome (PMS), adolescent depression, trichotillomania, dysthymia and substance abuse, as the selected substituted thiazol compounds are disclosed herein as inhibitors of DYRK1a. Further, the selected substituted thiazol compounds of the present invention enhance cognition as the selected substituted thiazol compounds are disclosed herein as inhibitors of DYRK1a.

The selected substituted thiazol compounds of the present invention are tested for their ability to treat depression in the model described in Porsolt et al., European Journal of Pharmacology, 51 (1978) 291-294.

The selected substituted thiazol compounds of the present invention treat the selected disease state of pancreatic cancer as the selected substituted thiazol compounds are disclosed herein as inhibitors of DYRK1b.

The selected substituted thiazol compounds of the present invention treat the selected disease states of bone resorption disease and osteoporosis as the selected substituted thiazol compounds are disclosed herein as inhibitors of DYRK2.

The selected substituted thiazol compounds of the present invention treat the selected disease states of sickle cell anemia and chronic kidney disease as the selected substituted thiazol compounds are known to be inhibitors of DYRK3.

As indicated by the references cited in the background of the invention, assays useful in determining if a compound is an inhibitor of one or more of DYRK1a, DYRK1b, DYRK2, DYRK3 and DYRK4 are known in the art. International Application No. PCT/US2006/019447, having an International filing date of May 18, 2006; International Publication Number WO 06/127458 and an International Publication date of Nov. 30, 2006 disclosed assays useful in determining whether a compound is an inhibitor of DYRK3 (indicated therein as YAK3). The selected substituted thiazol compounds of the present invention are tested for their ability to inhibit DYRK1a, DYRK1b, DYRK2, DYRK3 and DYRK4 according to assays well known to those skilled in the art.

By the term “selected substituted thiazol compound”, and derivatives thereof, as used herein to meant the compounds of Formula (IAA), Formula (IIAA), Formula (IIIAA), Structure (IAA), Structure (IIAA), Structure (IIIAAA), as described below, and the compounds that are the final products described in:

International Application No. PCT/US2005/006022, having an International filing date of Feb. 24, 2005; International Publication Number WO 05/082901 and an International Publication date of Sep. 9, 2005; and

International Application No. PCT/US2006/037090, having an International filing date of Sep. 22, 2006; International Publication Number WO 07/038,331 and an International Publication date of Apr. 5, 2007.

The compounds that are final products in both WO 05/082901 and WO 07/038,331 are useful in the present invention, these compounds are included herein by reference.

The compounds of Formula (IAA), Formula (IIAA), Formula (IIIAA), Structure (IAA), Structure (IIAA), Structure (IIIAAA) and the compounds that are final products in both WO 05/082901 and WO 07/038,331 can be prepared as indicated in their corresponding International Applications, the schemes and examples of which are incorporated by reference.

### Formula (IAA)

Included among the selected substituted thiazol compounds of the invention are compounds of Formula (I) as descried in International Application No. PCT/US2003/037658, having an International filing date of Nov. 18, 2003; International Publication Number WO 04/047760 and an International Publication date of Jun. 10, 2004. As descried in International Application No. PCT/US2003/037658, the compounds of Formula (I) (herein referred to as compounds of Formula (IAA)) have the following structure:

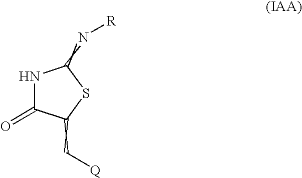

in which
R is C₃₋₆ cycloalkyl or naphtyl; or

### R is

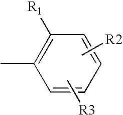 [0029] in which R1 is hydrogen, halogen, —C₁₋₆alkyl, —SC₁₋₆alkyl, —OC₁₋₆alkyl, —NO₂, —S(═O)—C₁₋₆alkyl, —OH, —CF₃, —CN, —CO₂H, —OCF₃, or —CO₂C₁₋₆alkyl; and R2 and R3 are independently hydrogen, halogen, —C₁₋₆ alkyl, —SC₁₋₆alkyl, —OC₁₋₆alkyl, —NO₂, —S(═O)—C₁₋₆alkyl, —OH, —CF₃, —CN, —CO₂H, —CO₂C₁₋₆alkyl, —CONH₂, —NH₂, —OCH₂(C═O)OH, —OCH₂CH₂OCH₃, —SO₂NH₂, —CH₂SO₂CH₃, —NH(C═NH)CH₃; or R2 and R3 can independently be a radical of the formula

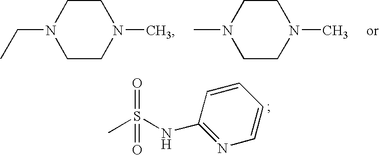

### Or

### R is

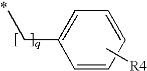 [0031] in which q is one or two; R4 is hydrogen, halogen, or —SO₂NH₂; or
R is —(CH₂)ₙ—NR^{k}R^{l} in which n is 2 or 3, and R^{k} and R^{l} are independently —C₁₋₆alkyl; or —NR^{k}R^{l} together form

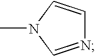

or

### R is

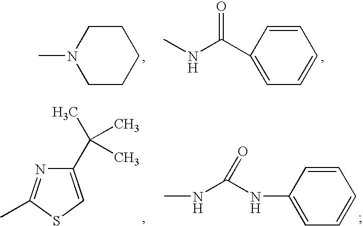

and

### Q is

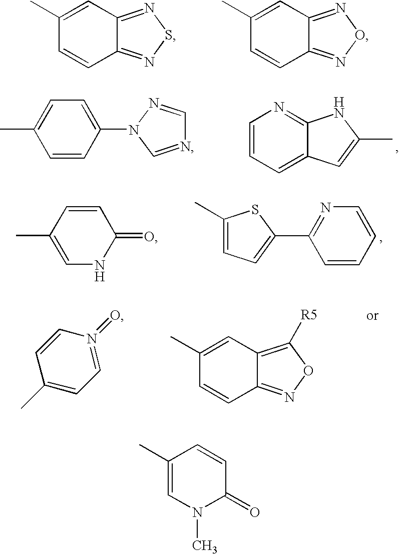

in which R5 is hydrogen, phenyl optionally substituted with up to three C₁₋₆ alkyl or halogen, or C₁₋₆ alkyl; or

### Q is

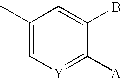

in which Y is CH; and A and B together are a part of

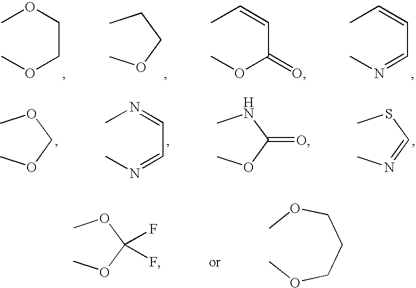

provided that ortho position to Y is N or O; or

### Q is

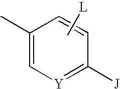 [0039] in which Y is N or CH; J is hydrogen, NH₂, OH or —OC₁₋₆alkyl; and L is hydrogen, NH₂, halogen, —NO₂, or —OC₁₋₆alkyl.

When referring to compounds of Formula (IAA), also included herein are salts, solvates, and physiologically functional derivatives thereof.

When referring to compounds of Formula (IAA),

the term “alkyl” refers to a straight or branched chain hydrocarbon. Furthermore, the term “C₁₋₆ alkyl” refers to an alkyl group as defined above containing at least 1, and at most 6, carbon atoms. Examples of such branched or straight chained “C₁₋₆ alkyl” groups include methyl, ethyl, n-propyl, isopropyl, isobutyl, n-butyl, t-butyl, n-pentyl, n-hexyl, and the like.

When referring to compounds of Formula (IAA),

the term “halogen” refers to fluorine (F), chlorine (Cl), bromine (Br), or iodine (I).

When referring to compounds of Formula (IAA),

the term “C₃₋₆ cycloalkyl” refers to a non-aromatic cyclic hydrocarbon ring having from three to six carbon atoms. Exemplary “C₃₋₆ cycloalkyl” groups include cyclopropyl, cyclobutyl, cyclopentyl, and cyclohexyl.

When referring to compounds of Formula (IAA),

the term “optionally” means that the subsequently described event(s) may or may not occur, and includes both event(s), which occur, and events that do not occur.

When referring to compounds of Formula (IAA),

the crisscrossed double bond indicated by the symbol [symbol] denotes Z and/or E stereochemistry around the double bond. In other words a compound of Formula I can be either in the Z or E stereochemistry around this double bond, or a compound of Formula I can be in a mixture of Z and E stereochemistry around the double bond. However, in Formula I, the preferred compounds have Z stereochemistry around the double bond to which radical Q is attached.

When referring to compounds of Formula (IAA),

the term “physiologically functional derivative” refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5th Edition, Vol 1: Principles and Practice, which is incorporated herein by reference to the extent that it teaches physiologically functional derivatives.

When referring to compounds of Formula (IAA),

the term “substituted” refers to substitution with the named substituent or substituents, multiple degrees of substitution being allowed unless otherwise stated.

Further definitions and descriptions of the compounds of Formula (IAA), including those directed to “solvate” and “tautomers'”, are found in International Application No. PCT/US2003/037658 and are incorporated herein by reference.

### Structure (IAA)

Included among the selected substituted thiazol compounds of the invention is the compound described in International Application No. PCT/US2006/022385, having an International filing date of Jun. 8, 2006; International Publication Number WO 06/135712 and an International Publication date of Dec. 21, 2006. As descried in International Application No. PCT/US2006/022385, the compound: (5Z)-5-(6-quinoxalinylmethylidene)-2-[(2,6-dichlorophenyl)amino]-1,3-thiazol-4(5H)-one is represented by Structure I (herein referred to as the compound of Structure (IAA):

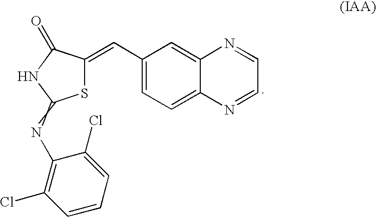

When referring to the compound of Structure (IAA), the free compound is contemplated and/or pharmaceutically acceptable salts, hydrates, solvates and pro-drugs thereof.

### Structure (IIAA)

As disclosed in International Application No. PCT/US2006/022385, the compound of Structure (IAA) is suitably in the form of a meglumine salt. The meglumine salt of the compound of Structure (IAA) is represented in International Application No. PCT/US2006/022385 by Structure II (herein referred to as the compound of Structure (IIAA):

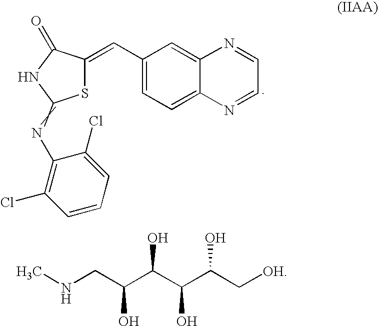

When referring to the compound of Structure (IIAA), the meglumine salt is contemplated and/or pharmaceutically acceptable hydrates, solvates and pro-drugs thereof.

When referring to the compounds of Structures IAA and IIAA, the crisscrossed double bond indicated by the symbol [symbol] denotes Z and/or E stereochemistry around the double bond. In other words, Structure (IAA), including Structure (IIAA), can be either in the Z or E stereochemistry around this double bond, or Structure (IAA), including Structure (IIAA), can be in a mixture of Z and E stereochemistry around the double bond.

Further definitions and descriptions of the compounds of Structure (IAA) and Structure (IIAA), including those directed to “tautomers'”, are found in International Application No. PCT/US2006/022385 and are incorporated herein by reference.

### Structure (IIIAA)

Included among the selected substituted thiazol compounds of the invention is the compound described in International Application No. PCT/US2006/022383, having an International filing date of Jun. 8, 2006; International Publication Number WO 06/133381 and an International Publication date of Dec. 14, 2006. As descried in International Application No. PCT/US2006/022383, the compound: (5Z)-5-(6-quinoxalinylmethylidene)-2-[(2,4,6-trichlorophenyl)amino]-1,3-thiazol-4(5H)-one is represented by Structure I (herein referred to as the compound of Structure (IIIAA):

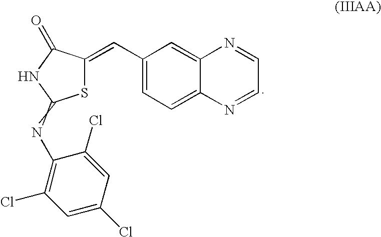

When referring to the compound of Structure (IIIAA), the free compound is contemplated and/or pharmaceutically acceptable salts, hydrates, solvates and pro-drugs thereof.

When referring to the compound of Structure IIIAA, the crisscrossed double bond indicated by the symbol [symbol] denotes Z and/or E stereochemistry around the double bond. In other words, Structure (IIIAA) can be either in the Z or E stereochemistry around this double bond, or Structure (IIIAA) can be in a mixture of Z and E stereochemistry around the double bond.

Further definitions and descriptions of the compound of Structure (IIIAA), including those directed to “tautomers'”, are found in International Application No. PCT/US2006/022383 and are incorporated herein by reference.

### Formula (IIAA)

Included among the selected substituted thiazol compounds of the invention are compounds of Formula (I) as descried in International Application No. PCT/US2006/017142, having an International filing date of May 3, 2006; International Publication Number WO 06/132739 and an International Publication date of Dec. 14, 2006. As descried in International Application No. PCT/US2006/017142, the compounds of Formula (I) (herein referred to as compounds of Formula (IIAA)) have the following structure:

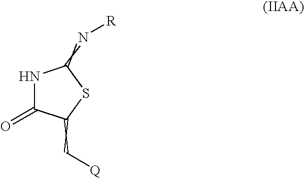

in which

R is selected form: aryl and substituted aryl; and

### Q is

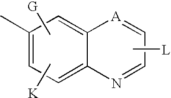

wherein

A is selected from CR⁵⁰ and N,
where R⁵⁰, G, K and L are each independently selected from the group consisting of: hydrogen, amino, alkylamine, substituted alkylamine, dialkylamine, substituted dialkylamine, hydroxy, alkylaminoalkyl, dialkylaminoalkyl, alkoxy, alkyl, substituted alkyl, aryl, substituted aryl, arylamine, substituted arylamine, halogen, cycloalkyl, substituted cycloalkyl, cycloalkyl containing from 1 to 4 heteroatoms, substituted cycloalkyl containing from 1 to 4 heteroatoms, —C(O)OR¹⁰, —C(O)NR¹¹R¹², oxo and cyano,
where, R¹⁰ is selected form hydrogen, C₁-C₄alkyl, aryl and trifluoromethyl, and R¹¹ and R¹² are independently selected form hydrogen, C₁-C₄alkyl, aryl and trifluoromethyl,
provided that at least one of G, K, L and R⁵⁰, when R⁵⁰ is present, is not hydrogen.

When referring to compounds of Formula (IIAA), the free compounds are contemplated and/or pharmaceutically acceptable salts, hydrates, solvates and pro-drugs thereof.

Included among the compounds useful in the present invention are:
(5Z)-2-[(2,6-Dichlorophenyl)amino]-5-{[3-(4-morpholinyl)-6-guinoxalinyl]methylidene}-1,3-thiazol-4(5H)-one;
7-{(Z)-[2-[(2,6-Dichlorophenyl)amino]-4-oxo-1,3-thiazol-5(4H)-ylidene]methyl}-2(1H)-quinoxalinone;
(5Z)-2-[(2,6-Dichlorophenyl)amino]-5-{[2-(methylamino)-6-quinolinyl]methylidene}-1,3-thiazol-4(5H)-one;
(5Z)-2-[(2,6-Dichlorophenyl)amino]-5-{[4-(dimethylamino)-6-quinolinyl]methylidene}-1,3-thiazol-4(5H)-one;
(5Z)-2-[(2,6-Dichlorophenyl)amino]-5-{[4-(4-methyl-1-piperazinyl)-6-quinolinyl]methylidene}-1,3-thiazol-4(5H)-one;
(5Z)-5-[(2-Chloro-6-quinolinyl)methylidene]-2-[(2,6-dichlorophenyl)amino]-1,3-thiazol-4(5H)-one;
(5Z)-2-[(2,6-dichlorophenyl)amino]-5-[(2-methyl-6-quinolinyl)methylidene]-1,3-thiazol-4(5H)-one;
(5Z)-5-[(7-Chloro-6-quinolinyl)methylidene]-2-[(2,6-dichlorophenyl)amino]-1,3-thiazol-4(5H)-one trifluoroacetate;
(5Z)-5-[(8-Chloro-6-quinolinyl)methylidene]-2-[(2,6-dichlorophenyl)amino]-1,3-thiazol-4(5H)-one, trifluoroacetate salt;
(5Z)-2-[(2-Chlorophenyl)amino]-5-[(8-methyl-6-quinolinyl)methylidene]-1,3-thiazol-4(5H)-one;
(5Z)-5-[(5-Chloro-6-quinolinyl)methylidene]-2-[(2,6-dichlorophenyl)amino]-1,3-thiazol-4(5H)-one;
(5Z)-2-[(2,6-Dichlorophenyl)amino]-5-[(2-pentyl-6-quinolinyl)methylidene]-1,3-thiazol-4(5H)-one;
6-{(Z)-[2-[(2,6-Dichlorophenyl)amino]-4-oxo-1,3-thiazol-5(4H)-ylidene]methyl}-2(1H)-quinolinone;
(5Z)-2-[(2,6-Dichlorophenyl)amino]-5-[(2-ethyl-6-quinolinyl)methylidene]-1,3-thiazol-4(5H)-one;
(5Z)-2-[(2,6-Dichlorophenyl)amino]-5-{[2-(methyloxy)-6-quinolinyl]methylidene}-1,3-thiazol-4(5H)-one;
(5Z)-2-[(2,6-Dichlorophenyl)amino]-5-{[2-(dimethylamino)-6-quinolinyl]methylidene}-1,3-thiazol-4(5H)-one;
(5Z)-2-[(2,6-Dichlorophenyl)amino]-5-[(4-hydroxy-6-quinolinyl)methylidene]-1,3-thiazol-4(5H)-one;
(5Z)-2-[(2,6-Dichlorophenyl)amino]-5-[(3-methyl-6-quinolinyl)methylidene]-1,3-thiazol-4(5H)-one;
(5Z)-2-[(2,6-Dichlorophenyl)amino]-5-{[3-(1-methylethyl)-6-quinolinyl]methylidene}-1,3-thiazol-4(5H)-one;
(5Z)-2-[(2,6-Dichlorophenyl)amino]-5-{[4-(methylamino)-6-quinolinyl]methylidene}-1,3-thiazol-4(5H)-one;
Ethyl-4-chloro-6-{(Z)-[2-[(2,6-dichlorophenyl)amino]-4-oxo-1,3-thiazol-5(4H)-ylidene]methyl}-3-quinolinecarboxylate;
(5Z)-5-[(4-Chloro-6-quinolinyl)methylidene]-2-[(2,6-dichlorophenyl)amino]-1,3-thiazol-4(5H)-one;
(5Z)-2-[(2,6-Dichlorophenyl)amino]-5-{[4-(methyloxy)-6-quinolinyl]methylidene}-1,3-thiazol-4(5H)-one;
(5Z)-2-[(2,6-Dichlorophenyl)amino]-5-{[4-(1-piperidinyl)-6-quinolinyl]methylidene}-1,3-thiazol-4(5H)-one trifluoroacetate;
(5Z)-2-[(2,6-Dichlorophenyl)amino]-5-{[4-(4-morpholinyl)-6-quinolinyl]methylidene}-1,3-thiazol-4(5H)-one;
(5Z)-2-[(2,6-Dichlorophenyl)amino]-5-[(8-fluoro-6-quinolinyl)methylidene]-1,3-thiazol-4(5H)-one;
(5Z)-2-[(2,6-Dichlorophenyl)amino]-5-({4-[(3,3-dimethylbutyl)amino]-6-quinolinyl}methylidene)-1,3-thiazol-4(5H)-one trifluoroacetate;
Ethyl-6-{(Z)-[2-[(2,6-dichlorophenyl)amino]-4-oxo-1,3-thiazol-5(4H)-ylidene]methyl}-3-quinolinecarboxylate;
6-{(Z)-[2-[(2,6-Dichlorophenyl)amino]-4-oxo-1,3-thiazol-5(4H)-ylidene]methyl}-3-quinolinecarboxylic acid;
6-{(Z)-[2-[(2,6-Dichlorophenyl)amino]-4-oxo-1,3-thiazol-5(4H)-ylidene]methyl}-3-quinolinecarboxamide;
(5Z)-5-({4-[(2-Cyclopropylethyl)amino]-6-quinolinyl}methylidene)-2-[(2,6-dichlorophenyl)amino]-1,3-thiazol-4(5H)-one trifluoroacetate;
(5Z)-2-[(2,6-Dichlorophenyl)amino]-5-{[4-(1-pyrrolidinyl)-6-quinolinyl]methylidene}-1,3-thiazol-4(5H)-one;
(5Z)-2-[(2,6-Dichlorophenyl)amino]-5-{[3-(hydroxymethyl)-6-quinolinyl]methylidene}-1,3-thiazol-4(5H)-one;
6-{(Z)-[2-[(2,6-Dichlorophenyl)amino]-4-oxo-1,3-thiazol-5(4H)-ylidene]methyl}-N-methyl-3-quinolinecarboxamide hydrochloride;
6-{(Z)-[2-[(2,6-Dichlorophenyl)amino]-4-oxo-1,3-thiazol-5(4H)-ylidene]methyl}-N,N-dimethyl-3-quinolinecarboxamide;
(5Z)-2-[(2,6-Dichlorophenyl)amino]-5-[(4-phenyl-6-quinolinyl)methylidene]-1,3-thiazol-4(5H)-one trifluoroacetate;
(5Z)-2-[(3-Chloro-2-biphenylyl)amino]-5-[(4-phenyl-6-quinolinyl)methylidene]-1,3-thiazol-4(5H)-one trifluoroacetate;
(5Z)-2-[(2,6-Dichlorophenyl)amino]-5-[(4-methyl-6-quinolinyl)methylidene]-1,3-thiazol-4(5H)-one;
(5Z)-2-[(2,6-Dichlorophenyl)amino]-5-{[4-(1-methylethyl)-6-quinolinyl]methylidene}-1,3-thiazol-4(5H)-one;
(5Z)-2-[(2,6-Dichlorophenyl)amino]-5-[(4-ethyl-6-quinolinyl)methylidene]-1,3-thiazol-4(5H)-one;
6-{(Z)-[2-[(2,6-Dichlorophenyl)amino]-4-oxo-1,3-thiazol-5(4H)-ylidene]methyl}-4-quinolinecarbonitrile;
(5Z)-2-[(2,6-Dichlorophenyl)amino]-5-{[4-(4-pyridinyl)-6-quinolinyl]methylidene}-1,3-thiazol-4(5H)-one;
(5Z)-2-[(2,6-Dichlorophenyl)amino]-5-{[4-(3-pyridinyl)-6-quinolinyl]methylidene}-1,3-thiazol-4(5H)-one;
Ethyl-6-{(Z)-[2-[(2,6-dichlorophenyl)amino]-4-oxo-1,3-thiazol-5(4H)-ylidene]methyl}-4-(4-morpholinyl)-3-quinolinecarboxylate;
Ethyl-6-{(Z)-[2-[(2,6-dichlorophenyl)amino]-4-oxo-1,3-thiazol-5(4H)-ylidene]methyl}-4-(4-methyl-1-piperazinyl)-3-quinolinecarboxylate;
6-{(Z)-[2-[(2,6-Dichlorophenyl)amino]-4-oxo-1,3-thiazol-5(4H)-ylidene]methyl}-4-(4-morpholinyl)-3-quinolinecarboxamide trifluoroacetate;
N-{4-Chloro-3-[((5Z)-5-{[4-(4-morpholinyl)-6-quinolinyl]methylidene}-4-oxo-4,5-dihydro-1,3-thiazol-2-yl)amino]phenyl}cyclobutanecarboxamide;
N-{4-Chloro-3-[((5Z)-5-{[4-(4-methyl-1-piperazinyl)-6-quinolinyl]methylidene}-4-oxo-4,5-dihydro-1,3-thiazol-2-yl)amino]phenyl}cyclobutanecarboxamide;
6-{(Z)-[2-[(2,6-Dichlorophenyl)amino]-4-oxo-1,3-thiazol-5(4H)-ylidene]methyl}-4-(4-methyl-1-piperazinyl)-3-quinolinecarboxamide trifluoroacetate;
(5Z)-2-[(2,6-Dichlorophenyl)amino]-5-{[4-(1-piperazinyl)-6-quinolinyl]methylidene}-1,3-thiazol-4(5H)-one;
N-{4-Chloro-3-[((5Z)-4-oxo-5-{[4-(1-piperazinyl)-6-quinolinyl]methylidene}-4,5-dihydro-1,3-thiazol-2-yl)amino]phenyl}cyclobutanecarboxamide;
Ethyl-6-{(Z)-[2-[(2,6-dichlorophenyl)amino]-4-oxo-1,3-thiazol-5(4H)-ylidene]methyl}-4-(dimethylamino)-3-quinolinecarboxylate;
Ethyl-6-{(Z)-[2-[(2,6-dichlorophenyl)amino]-4-oxo-1,3-thiazol-5(4H)-ylidene]methyl}-4-(methylamino)-3-quinolinecarboxylate;
Ethyl-6-{(Z)-[2-({2-chloro-5-[(cyclobutylcarbonyl)amino]phenyl}amino)-4-oxo-1,3-thiazol-5(4H)-ylidene]methyl}-4-(dimethylamino)-3-quinolinecarboxylate trifluoroacetate;
Ethyl-6-{(Z)-[2-({2-chloro-5-[(cyclobutylcarbonyl)amino]phenyl}amino)-4-oxo-1,3-thiazol-5(4H)-ylidene]methyl}-4-(methylamino)-3-quinolinecarboxylate;
Ethyl-6-{(Z)-[2-({2-chloro-5-[(cyclobutylcarbonyl)amino]phenyl}amino)-4-oxo-1,3-thiazol-5(4H)-ylidene]methyl}-4-(4-morpholinyl)-3-quinolinecarboxylate trifluoroacetate;
Ethyl-6-{(Z)-[2-[(2,6-dichlorophenyl)amino]-4-oxo-1,3-thiazol-5(4H)-ylidene]methyl}-4-(4-pyridinyl)-3-quinolinecarboxylate trifluoroacetate;
Ethyl-6-{(Z)-[2-({2-chloro-5-[(cyclobutylcarbonyl)amino]phenyl}amino)-4-oxo-1,3-thiazol-5(4H)-ylidene]methyl}-4-(4-pyridinyl)-3-quinolinecarboxylate trifluoroacetate;
6-{(Z)-[2-[(2,6-Dichlorophenyl)amino]-4-oxo-1,3-thiazol-5(4H)-ylidene]methyl}-4-(4-pyridinyl)-3-quinolinecarboxamide trifluoroacetate;
6-{(Z)-[2-[(2,6-Dichlorophenyl)amino]-4-oxo-1,3-thiazol-5(4H)-ylidene]methyl}-4-quinolinecarboxylic acid trifluoroacetate;
6-{(Z)-[2-[(2,6-Dichlorophenyl)amino]-4-oxo-1,3-thiazol-5(4H)-ylidene]methyl}-4-quinolinecarboxamide trifluoroacetate;
Methyl-6-{(Z)-[2-[(2,6-dichlorophenyl)amino]-4-oxo-1,3-thiazol-5(4H)-ylidene]methyl}-4-quinolinecarboxylate trifluoroacetate;
6-{(Z)-[2-[(2,6-Dichlorophenyl)amino]-4-oxo-1,3-thiazol-5(4H)-ylidene]methyl}-N-methyl-4-quinolinecarboxamide trifluoroacetate;
6-{(Z)-[2-[(2,6-Dichlorophenyl)amino]-4-oxo-1,3-thiazol-5(4H)-ylidene]methyl}-N,N-dimethyl-4-quinolinecarboxamide trifluoroacetate;
(5Z)-2-[(2,6-Dichlorophenyl)amino]-5-{[4-(4-morpholinylcarbonyl)-6-quinolinyl]methylidene}-1,3-thiazol-4(5H)-one trifluoroacetate;
(5Z)-2-[(2,6-Dichlorophenyl)amino]-5-({4-[(4-methyl-1-piperazinyl)carbonyl]-6-quinolinyl}methylidene)-1,3-thiazol-4(5H)-one trifluoroacetate;
(5Z)-2-[(2,6-Dichlorophenyl)amino]-5-{[3-(dimethylamino)-6-quinoxalinyl]methylidene}-1,3-thiazol-4(5H)-one;
(5Z)-2-[(2,6-Dichlorophenyl)amino]-5-({3-[2-(dimethylamino)ethyl]-6-quinoxalinyl}methylidene)-1,3-thiazol-4(5H)-one;
Ethyl-6-{(Z)-[2-({2-chloro-5-[(2-methylpropanoyl)amino]phenyl}amino)-4-oxo-1,3-thiazol-5(4H)-ylidene]methyl}-4-(4-pyridinyl)-3-quinolinecarboxylate;
(5Z)-2-[(2,6-Dichlorophenyl)amino]-5-{[3-(methyloxy)-6-quinoxalinyl]methylidene}-1,3-thiazol-4(5H)-one;
(5Z)-2-[(2,6-Dichlorophenyl)amino]-5-{[3-(4-methyl-1-piperazinyl)-6-quinoxalinyl]methylidene}-1,3-thiazol-4(5H)-one;
(5Z)-2-[(2,6-Dichlorophenyl)amino]-5-[(3-methyl-6-quinoxalinyl)methylidene]-1,3-thiazol-4(5H)-one;
(5Z)-2-[(2,6-Dichlorophenyl)amino]-5-[(3-phenyl-6-quinoxalinyl)methylidene]-1,3-thiazol-4(5H)-one;
(5Z)-2-[(2,6-Dichlorophenyl)amino]-5-({3-[(2-hydroxyethyl)(methyl)amino]-6-quinoxalinyl}methylidene)-1,3-thiazol-4(5H)-one;
(5Z)-2-[(2,6-Dichlorophenyl)amino]-5-({3-[[2-(dimethylamino)ethyl](methyl)amino]-6-quinoxalinyl}methylidene)-1,3-thiazol-4(5H)-one;
(5Z)-2-[(2,6-Dichlorophenyl)amino]-5-{[3-(phenylamino)-6-quinoxalinyl]methylidene}-1,3-thiazol-4(5H)-one;
N-{4-Chloro-3-[((5Z)-5-{[3-(4-morpholinyl)-6-quinoxalinyl]methylidene}-4-oxo-4,5-dihydro-1,3-thiazol-2-yl)amino]phenyl}cyclobutanecarboxamide;
(5Z)-5-[(3-Amino-6-quinoxalinyl)methylidene]-2-[(2,6-dichlorophenyl)amino]-1,3-thiazol-4(5H)-one;
N-{4-Chloro-3-[((5Z)-5-{[4-(4-methyl-1-piperazinyl)-6-quinolinyl]methylidene}-4-oxo-4,5-dihydro-1,3-thiazol-2-yl)amino]phenyl}-2-methylpropanamide trifluoroacetate;
N-{4-Chloro-3-[((5Z)-5-{[4-(4-morpholinyl)-6-quinolinyl]methylidene}-4-oxo-4,5-dihydro-1,3-thiazol-2-yl)amino]phenyl}-2-methylpropanamide trifluoroacetate;
N-{4-Chloro-3-[((5Z)-5-{[4-(4-methyl-1-piperazinyl)-6-quinolinyl]methylidene}4-oxo-4,5-dihydro-1,3-thiazol-2-yl)amino]phenyl}ethanesulfonamide trifluoroacetate;
N-{4-Chloro-3-[((5Z)-5-{[4-(4-morpholinyl)-6-quinolinyl]methylidene}-4-oxo-4,5-dihydro-1,3-thiazol-2-yl)amino]phenyl}ethanesulfonamide trifluoroacetate;
Ethyl-6-{(Z)-[2-({2-chloro-5-[(2-methylpropanoyl)amino]phenyl}amino)-4-oxo-1,3-thiazol-5(4H)-ylidene]methyl}-4-(4-morpholinyl)-3-quinolinecarboxylate trifluoroacetate;
Ethyl-6-{(Z)-[2-({2-chloro-5-[(2-methylpropanoyl)amino]phenyl}amino)-4-oxo-1,3-thiazol-5(4H)-ylidene]methyl}-3-quinolinecarboxylate trifluoroacetate;
Ethyl-6-{(Z)-[2-({2-chloro-5-[(2-methylpropanoyl)amino]phenyl}amino)-4-oxo-1,3-thiazol-5(4H)-ylidene]methyl}-4-(4-methyl-1-piperazinyl)-3-quinolinecarboxylate trifluoroacetate; and
Ethyl-6-{(Z)-[2-({2-chloro-5-[(cyclobutylcarbonyl)amino]phenyl}amino)-4-oxo-1,3-thiazol-5(4H)-ylidene]methyl}-3-quinolinecarboxylate trifluoroacetate, and/or a pharmaceutically acceptable salt, hydrate, solvate or pro-drug thereof.

When referring to compounds of Formula (IIAA), the term “aryl” is meant a cyclic or polycyclic aromatic ring containing from 1 to 14 carbon atoms and optionally containing from one to five heteroatoms, provided that when the number of carbon atoms is 1 the aromatic ring contains at least four heteroatoms, when the number of carbon atoms is 2 the aromatic ring contains at least three heteroatoms, when the number of carbons is 3 the aromatic ring contains at least two heteroatoms and when the number of carbon atoms is 4 the aromatic ring contains at least one heteroatom.

When referring to compounds of Formula (IIAA), the term “C₁-C₁₂aryl” is meant phenyl, naphthalene, 3,4-methylenedioxyphenyl, pyridine, biphenyl, quinoline, pyrimidine, quinazoline, thiophene, thiazole, furan, pyrrole, pyrazole, imidazole, indole, indene, pyrazine, 1,3-dihydro-2H-benzimidazol, benzimidazol, benzothiohpene, tetrahydrobenzothiohpene and tetrazole.

When referring to compounds of Formula (IIAA), the term “substituted” is meant that the subject chemical moiety has one or more substituents selected from the group consisting of: aryl,

aryl substituted with one or more subsititents selected from alkyl, hydroxy, alkoxy, oxo, C₁-C₁₂aryl optionally substituted with one or more substituents selected from hydroxy, alkoxy oxo, cyano, amino, alkylamino, dialkylamino, alkyl and alkoxy, trifluoromethyl, —SO₂NR²¹R²², N-acylamino, —CO₂R²⁰, and halogen, cycloalkyl substituted with one or more subsititents selected from alkyl, hydroxy, alkoxy, trifluoromethyl, —SO₂NR²¹R²², amino, —CO₂R²⁰, N-acylamino and halogen,
cycloalkyl containing from 1 to 4 heteroatoms substituted with one or more subsititents selected from alkyl, hydroxy, alkoxy, —SO₂NR²¹R²², amino, —CO₂R²⁰, trifluoromethyl, N-acylamino and halogen, alkoxy substituted with one or more substituents selected form alkyl, —CO₂H, hydroxyl, C₁-C₁₂aryl, alkoxy, amino and halogen,
cycloalkyl, cycloalkyl containing from 1 to 4 heteroatoms, C₁-C₄alkylcycloalkyl containing from 1 to 3 heteroatoms C₁-C₄alkyl, —C(O)NHS(O)₂R²⁰, —(CH₂)_{g}NR²³S(O)₂R²⁰, hydroxyalkyl, alkoxy, —(CH₂)_{g}NR²¹R²², —C(O)NR²¹R²², —S(O)₂NR²¹R²², —(CH₂)_{g}N(R²⁰)C(O)ₙR²⁰, —(CH₂)_{g}N═C(H)R²⁰, —C(O)R²⁰, acyloxy, —SC₁-C₆alkyl, alkyl, —OCF₃, amino, hydroxy, alkylamino, acetamide, aminoalkyl, aminoalkoxy, alkylaminoalkoxy, dialkylaminoalkoxy, alkoxyalkylamide, alkoxyC₁-C₁₂aryl, C₁-C₁₂aryl, C₁-C₁₂arylalkyl, dialkylamino, N-acylamino, aminoalkylN-acylamino, —(CH₂)_{g}C(O)OR²⁰, —(CH₂)_{g}S(O)ₙR²³, nitro, cyano, oxo, halogen, trifluoromethyloxy and trifluoromethyl;
where g is 0 to 6, n is 0 to 2, R²³ is hydrogen or alkyl, each R²⁰ is independently selected form hydrogen, alkyl, C₁-C₆alkyloxyC₁-C₆alkyl, C₁-C₄alkylC(O)OC₁-C₄alkyl, amino, alkylamino, dialkylamino, aminoC₁-C₆alkyl, alkylaminoC₁-C₆alkyl, dialkylaminoC₁-C₆alkyl, —C(O)OH, alkoxy, aryloxy, arylamino, diarylamino, arylalkylamino, aryl, aryl substituted with one or more substituents selected from oxo, hydroxyl and alkyl, arylC₁-C₄alkyl optionally substituted with one or more substituents selected from oxo, hydroxy, halogen, alkoxy and alkyl, —CH₂C(O)cycloalkyl containing from 1 to 4 heteroatoms, cycloalkylC₁-C₄alkyl, C₁-C₄alkyl substituted with cycloalkyl containing from 1 to 4 heteroatoms, cycloalkyl, cycloalkyl substituted with one or more substituents selected from oxo, hydroxyl and alkyl, cycloalkyl containing from 1 to 4 heteroatoms, cycloalkyl containing from 1 to 4 heteroatoms substituted with one or more substituents selected from oxo, hydroxyl and alkyl, and trifluoromethyl, and R²¹ and R²² are independently selected form hydrogen, alkyl, C₁-C₆alkyl substituted with one of more substituents selected from hydroxy, amino, ═NH, and EN, —S(O)₂aryl, —S(O)₂alkyl, C₁-C₁₂aryl, cycloalkyl containing from 1 to 4 heteroatoms, cycloalkyl containing from 1 to 4 heteroatoms substituted with one or more substituents selected from oxo, hydroxy, and alkyl, cycloalkyl, cycloalkyl substituted with one or more substituents selected from oxo, hydroxy, and alkyl, arylC₁-C₆alkyl optionally substituted with one or more substituents selected from oxo, hydroxy, and alkyl, cycloalkyl containing from 1 to 4 heteroatoms optionally substituted with one or more substituents selected from oxo, hydroxyl and alkyl, C₁-C₆alkoxy, C₁-C₄alkyloxyC₁-C₄alkyl, aryl and trifluoromethyl.

When referring to compounds of Formula (IAA), suitably, the term “substituted” means that the subject chemical moiety has from one to five of the indicated substituents, suitably from one to three of the indicated substituents, suitably one or two of the indicated substituents.

When referring to compounds of Formula (IIAA), the term “cycloalkyl” means a nonaromatic, unsaturated or saturated, cyclic or polycyclic C₃-C₁₂.

When referring to compounds of Formula (IIAA), suitably cycloalkyl and substituted cycloalkyl substituents include: cyclohexyl, aminocyclohexyl, cyclobutyl, aminocyclobutyl, 4-hydroxy-cyclohexyl, 2-ethylcyclohexyl, propyl4-methoxycyclohexyl, 4-methoxycyclohexyl, 4-carboxycyclohexyl, cyclopropyl, aminocyclopentyl, and cyclopentyl.

When referring to compounds of Formula (IIAA), the term “cycloalkyl containing from 1 to 4 heteroatoms” and the term “cycloalkyl containing from 1 to 3 heteroatoms” means a nonaromatic, unsaturated or saturated, cyclic or polycyclic ring containing from 1 to 12 carbons and containing from one to four heteroatoms or from one to three heteroatoms (respectively), provided that when the number of carbon atoms is 1 the aromatic ring contains at least four heteroatoms (applicable only where “cycloalkyl containing from 1 to 4 heteroatoms” is indicated), when the number of carbon atoms is 2 the aromatic ring contains at least three heteroatoms, when the number of carbon atoms is 3 the nonaromatic ring contains at least two heteroatoms and when the number of carbon atoms is 4 the nonaromatic ring contains at least one heteroatom.

When referring to compounds of Formula (IIAA), suitable examples of cycloalkyl containing from 1 to 4 heteroatoms, cycloalkyl containing from 1 to 3 heteroatoms, substituted cycloalkyl containing from 1 to 4 heteroatoms and substituted cycloalkyl containing from 1 to 3 heteroatoms include: piperidine, piperazine, pyrrolidine, 3-methylaminopyrrolidine, piperazinly, tetrazole, hexahydrodiazepine and morpholine.

By the term “acyloxy” as used herein is meant —OC(O)alkyl where alkyl is as described for Formula (IIAA). Examples of acyloxy substituents as used herein include: —OC(O)CH₃, —OC(O)CH(CH₃)₂ and —OC(O)(CH₂)₃CH₃.

When referring to compounds of Formula (IIAA), the term “N-acylamino” means —N(H)C(O)alkyl, where alkyl is as described herein. Examples of N-acylamino substituents include: —N(H)C(O)CH₃, —N(H)C(O)CH(CH₃)₂ and —N(H)C(O)(CH₂)₃CH₃.

When referring to compounds of Formula (IIAA), the term “aryloxy” means —Oaryl where aryl is phenyl, naphthyl, 3,4-methylenedioxyphenyl, pyridyl or biphenyl optionally substituted with one or more substituents selected from the group consisting of: alkyl, hydroxyalkyl, alkoxy, trifluoromethyl, acyloxy, amino, N-acylamino, hydroxy, —(CH₂)_{g}C(O)OR²⁵, —S(O)ₙR²⁵, nitro, cyano, halogen and protected —OH, where g is 0-6, R²⁵ is hydrogen or alkyl, and n is 0-2. Examples of aryloxy substituents include: phenoxy, 4-fluorophenyloxy and biphenyloxy.

When referring to compounds of Formula (IIAA), the term “heteroatom” means oxygen, nitrogen or sulfur.

When referring to compounds of Formula (IIAA), the term “halogen” means a substituent selected from bromide, iodide, chloride and fluoride.

When referring to compounds of Formula (IIAA), the term “alkyl” and derivatives thereof and in all carbon chains as for Formula (IIAA), including alkyl chains defined by the term “—(CH₂)ₙ”, “—(CH₂)ₘ” and the like, is meant a linear or branched, saturated or unsaturated hydrocarbon chain, and unless otherwise defined, the carbon chain will contain from 1 to 12 carbon atoms.

When referring to compounds of Formula (IIAA), examples of alkyl and substituted alkyl substituents include:

—CH₃, —CH₂—CH₃, —CH₂—CH₂—CH₃, —CH(CH₃)₂, —CH₂—CH₂—C(CH₃)₃, —CH₂—CF₃, —C≡C—C(CH₃)₃, —C≡C—CH₂—OH, cyclopropylmethyl, —CH₂—C(CH₃)₂—CH₂—NH₂, —C≡C—C₆H₅, —C≡C—C(CH₃)₂—OH, —CH₂—CH(OH)—CH(OH)—CH(OH)—CH(OH)—CH₂—OH, piperidinylmethyl, methoxyphenylethyl, —C(CH₃)₃, —(CH₂)₃—CH₃, —CH₂—CH(CH₃)₂, —CH(CH₃)—CH₂—CH₃, —CH═CH₂, and —C≡C—CH₃.

When referring to compounds of Formula (IIAA), the crisscrossed double bond indicated by the symbol [symbol] denotes Z and/or E stereochemistry around the double bond. In other words a compound of Formula (IIAA) can be either in the Z or E stereochemistry around this double bond, or a compound of Formula IIAA can be in a mixture of Z and E stereochemistry around the double bond

Further definitions and descriptions of the compounds of Formula (IIAA), including those directed to “tautomers'”, are found in International Application No. PCT/US2006/017142 and are incorporated herein by reference.

### Formula (IIIAAA)

Included among the selected substituted thiazol compounds of the invention are compounds of Formula (I) as descried in International Application No. PCT/US2006/019447, having an International filing date of May 18, 2006; International Publication Number WO 06/127458 and an International Publication date of Nov. 30, 2006. As descried in International Application No. PCT/US2006/019447, the compounds of Formula (I) (herein referred to as compounds of Formula (IIIAA)) have the following structure:

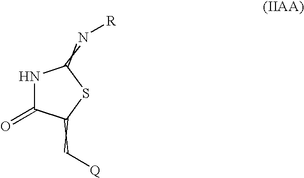

in which

R is selected form: aryl and substituted aryl; and

### Q is

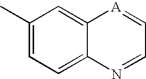

wherein

A is selected from CH and N;

and/or pharmaceutically acceptable salts, hydrates, solvates and pro-drugs thereof, provided that when R is

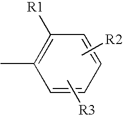

R¹ is not hydrogen, halogen, —C₁₋₆alkyl, —SC₁₋₆alkyl, —OC₁₋₆alkyl, —NO₂, —S(═O)—C₁₋₆alkyl, —OH, —CF₃, —CN, —CO₂H, —OCF₃, or —CO₂C₁₋₆alkyl, when R² and R³ are independently selected from: hydrogen, halogen, —C₁₋₆ alkyl, —SC₁₋₆alkyl, —OC₁₋₆alkyl, —NO₂, —S(═O)—C₁₋₆alkyl, —OH, —CF₃, —CN, —CO₂H, —CO₂C₁₋₆alkyl, —CONH₂, —NH₂, —OCH₂(C═O)OH, —OCH₂CH₂OCH₃, —SO₂NH₂, —CH₂SO₂CH₃, and —NH(C═NH)CH₃, and

further provided that R is not naphthyl.

Suitably, the compounds of Formula (IIIAA) contain the further proviso that R is not t-butylthiazol.

Suitably, the compounds of Formula (IIIAA) contain the further proviso that R is not t-butylthiazol and the further proviso that R¹ is not hydrogen, halogen, —C₁₋₆alkyl, —SC₁₋₆alkyl, —OC₁₋₆alkyl, —NO₂, —S(═O)—C₁₋₆alkyl, —OH, —CF₃, —CN, —CO₂H, —OCF₃, or —CO₂Cl_{—}6alkyl when R² and R³ are independently selected from:

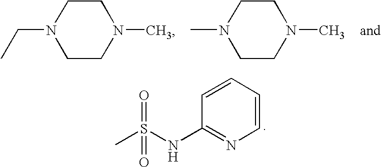

Included among the compounds useful in the present invention are:
(5Z)-2-[(2-Chloro-3-pyridinyl)amino]-5-(6-quinolinylmethylidene)-1,3-thiazol-4(5H)-one;
(5Z)-2-{[2-Chloro-5-(2-pyridinyl)phenyl]amino}-5-(6-quinolinylmethylidene)-1,3-thiazol-4(5H)-one;
N-(4-Chloro-3-{[(5Z)-4-oxo-5-(6-quinolinylmethylidene)-4,5-dihydro-1,3-thiazol-2-yl]amino}phenyl)cyclobutanecarboxamide;
N-(4-chloro-3-{[(5Z)-4-oxo-5-(6-quinoxalinylmethylidene)-4,5-dihydro-1,3-thiazol-2-yl]amino}phenyl)-2-methylpropanamide;
N-(4-chloro-3-{[(5Z)-4-oxo-5-(6-quinolinylmethylidene)-4,5-dihydro-1,3-thiazol-2-yl]amino}phenyl)methanesulfonamide;
N-[(4-Chloro-3-{[(5Z)-4-oxo-5-(6-quinolinylmethylidene)-4,5-dihydro-1,3-thiazol-2-yl]amino}phenyl)methyl]-2-(methyloxy)acetamide;
(5Z)-2-[(2-Chloro-5-{[(1-methylethyl)amino]methyl}phenyl)amino]-5-(6-quinolinylmethylidene)-1,3-thiazol-4(5H)-one;
(2Z,5Z)-2-[(5-Chloro-1H-benzimidazol-6-yl)imino]-5-(quinolin-6-ylmethylene)-1,3-thiazolidin-4-one;
4-Chloro-N-cyclobutyl-3-{[(5Z)-4-oxo-5-(6-quinolinylmethylidene)-4,5-dihydro-1,3-thiazol-2-yl]amino}benzenesulfonamide;
(5Z)-2-(4-Pyrimidinylamino)-5-(6-quinolinylmethylidene)-1,3-thiazol-4(5H)-one;
(5Z)-2-(1H-Pyrazol-3-ylamino)-5-(6-quinolinylmethylidene)-1,3-thiazol-4(5H)-one;
(5Z)-2-(4-Pyridinylamino)-5-(6-quinolinylmethylidene)-1,3-thiazol-4(5H)-one;
(5Z)-2-(2-Quinolinylamino)-5-(6-quinolinylmethylidene)-1,3-thiazol-4(5H)-one;
(5Z)-2-[(4-Methyl-2-pyridinyl)amino]-5-(6-quinolinylmethylidene)-1,3-thiazol-4(5H)-one;
(5Z)-2-[(3-Methyl-2-pyridinyl)amino]-5-(6-quinolinylmethylidene)-1,3-thiazol-4(5H)-one;
(5Z)-2-[(6-Methyl-2-pyridinyl)amino]-5-(6-quinolinylmethylidene)-1,3-thiazol-4(5H)-one;
(5Z)-2-[(5-Iodo-2-pyridinyl)amino]-5-(6-quinolinylmethylidene)-1,3-thiazol-4(5H)-one;
(5Z)-2-(1H-Benzimidazol-2-ylamino)-5-(6-quinolinylmethylidene)-1,3-thiazol-4(5H)-one;
N-(3-{[(5Z)-4-Oxo-5-(6-quinolinylmethylidene)-4,5-dihydro-1,3-thiazol-2-yl]amino}phenyl)acetamide;
1,1-Dimethylethyl (2-{[(5Z)-4-oxo-5-(6-quinolinylmethylidene)-4,5-dihydro-1,3-thiazol-2-yl]amino}phenyl)carbamate;
N-(2-{[(5Z)-4-Oxo-5-(6-quinolinylmethylidene)-4,5-dihydro-1,3-thiazol-2-yl]amino}phenyl)acetamide;
(5Z)-2-(2-Pyrimidinylamino)-5-(6-quinolinylmethylidene)-1,3-thiazol-4(5H)-one;
(5Z)-2-(3-Quinolinylamino)-5-(6-quinolinylmethylidene)-1,3-thiazol-4(5H)-one;
(5Z)-2-[(1-Methyl-1H-indol-2-yl)amino]-5-(6-quinolinylmethylidene)-1,3-thiazol-4(5H)-one;
(5Z)-2-[(5-Chloro-2-pyridinyl)amino]-5-(6-quinolinylmethylidene)-1,3-thiazol-4(5H)-one;
1,1-Dimethylethyl [(2-{[(5Z)-4-oxo-5-(6-quinolinylmethylidene)-4,5-dihydro-1,3-thiazol-2-yl]amino}phenyl)methyl]carbamate;
(5Z)-2-{[2-Chloro-6-(trifluoromethyl)-3-pyridinyl]amino}-5-(6-quinolinylmethylidene)-1,3-thiazol-4(5H)-one;
N-(4-{[(5Z)-4-Oxo-5-(6-quinolinylmethylidene)-4,5-dihydro-1,3-thiazol-2-yl]amino}phenyl)acetamide;
(5Z)-2-{[6-(Methyloxy)-4-pyrimidinyl]amino}-5-(6-quinolinylmethylidene)-1,3-thiazol-4(5H)-one;
3-Methyl-N-(2-{[(5Z)-4-oxo-5-(6-quinolinylmethylidene)-4,5-dihydro-1,3-thiazol-2-yl]amino}phenyl)butanamide;
N-(4-Chloro-3-{[(5Z)-4-oxo-5-(6-quinolinylmethylidene)-4,5-dihydro-1,3-thiazol-2-yl]amino}phenyl)-3-methylbutanamide;
(5Z)-2-[(4-Methyl-1,3-thiazol-2-yl)amino]-5-(6-quinolinylmethylidene)-1,3-thiazol-4(5H)-one;
Ethyl 2-{[(5Z)-4-oxo-5-(6-quinolinylmethylidene)-4,5-dihydro-1,3-thiazol-2-yl]amino}-4,5,6,7-tetrahydro-1-benzothiophene-3-carboxylate;
(5Z)-2-(2-Biphenylylamino)-5-(6-quinolinylmethylidene)-1,3-thiazol-4(5H)-one;
(5Z)-2-{[4′-(Methyloxy)-2-biphenylyl]amino}-5-(6-quinolinylmethylidene)-1,3-thiazol-4(5H)-one;
(5Z)-2-{[4′-(Dimethylamino)-2-biphenylyl]amino}-5-(6-quinolinylmethylidene)-1,3-thiazol-4(5H)-one;
2′-{[(5Z)-4-Oxo-5-(6-quinolinylmethylidene)-4,5-dihydro-1,3-thiazol-2-yl]amino}-3-biphenylcarbonitrile;
(5Z)-2-{[2-(1,3-Benzodioxol-5-yl)phenyl]amino}-5-(6-quinolinylmethylidene)-1,3-thiazol-4(5H)-one;
Ethyl 1-methyl-5-{[(5Z)-4-oxo-5-(6-quinolinylmethylidene)-4,5-dihydro-1,3-thiazol-2-yl]amino}-1H-pyrazole-4-carboxylate;
(5Z)-2-{[2-(4-Pyridinyl)phenyl]amino}-5-(6-quinolinylmethylidene)-1,3-thiazol-4(5H)-one;
(5Z)-2-{[2-Chloro-5-(hydroxymethyl)phenyl]amino}-5-(6-quinolinylmethylidene)-1,3-thiazol-4(5H)-one;
1,1-Dimethylethyl [(4-chloro-3-{[(5Z)-4-oxo-5-(6-quinolinylmethylidene)-4,5-dihydro-1,3-thiazol-2-yl]amino}phenyl)methyl]carbamate;
(5Z)-2-{[5-(Aminomethyl)-2-chlorophenyl]amino}-5-(6-quinolinylmethylidene)-1,3-thiazol-4(5H)-one;
N-(4-Chloro-3-{[(5Z)-4-oxo-5-(6-quinolinylmethylidene)-4,5-dihydro-1,3-thiazol-2-yl]amino}phenyl)propanamide;
N-(4-Chloro-3-{[(5Z)-4-oxo-5-(6-quinolinylmethylidene)-4,5-dihydro-1,3-thiazol-2-yl]amino}phenyl)-2-(methyloxy)acetamide;
N-(4-Chloro-3-{[(5Z)-4-oxo-5-(6-quinolinylmethylidene)-4,5-dihydro-1,3-thiazol-2-yl]amino}phenyl)-2-(2-thienyl)acetamide;
N-[(4-Chloro-3-{[(5Z)-4-oxo-5-(6-quinolinylmethylidene)-4,5-dihydro-1,3-thiazol-2-yl]amino}phenyl)methyl]acetamide;
N-[(4-Chloro-3-{[(5Z)-4-oxo-5-(6-quinolinylmethylidene)-4,5-dihydro-1,3-thiazol-2-yl]amino}phenyl)methyl]-2-methylpropanamide;
N˜1˜-(4-Chloro-3-{[(5Z)-4-oxo-5-(6-quinolinylmethylidene)-4,5-dihydro-1,3-thiazol-2-yl]amino}phenyl)-N˜2˜, N˜2™dimethylglycinamide;
N-[(4-Chloro-3-{[(5Z)-4-oxo-5-(6-quinolinylmethylidene)-4,5-dihydro-1,3-thiazol-2-yl]amino}phenyl)methyl]urea;
N-[(4-Chloro-3-{[(5Z)-4-oxo-5-(6-quinolinylmethylidene)-4,5-dihydro-1,3-thiazol-2-yl]amino}phenyl)methyl]-2-hydroxyacetamide;
N˜1˜-[4(4-Chloro-3-{[(5Z)-4-oxo-5-(6-quinolinylmethylidene)-4,5-dihydro-1,3-thiazol-2-yl]amino}phenyl)methyl]-N˜2˜,N˜2˜dimethylglycinamide;
1,1-Dimethylethyl (2-{[(4-chloro-3-{[(5Z)-4-oxo-5-(6-quinolinylmethylidene)-4,5-dihydro-1,3-thiazol-2-yl]amino}phenyl)methyl]amino}-2-oxoethyl)carbamate;
4-{[(4-Chloro-3-{[(5Z)-4-oxo-5-(6-quinolinylmethylidene)-4,5-dihydro-1,3-thiazol-2-yl]amino}phenyl)methyl]amino}-4-oxobutanoic acid;
(5Z)-2-{[3-(1,3-Oxazol-4-yl)phenyl]amino}-5-(6-quinolinylmethylidene)-1,3-thiazol-4(5H)-one;
(5Z)-2-[(1-Ethyl-1H-pyrazol-5-yl)amino]-5-(6-quinolinylmethylidene)-1,3-thiazol-4(5H)-one;
N˜1˜-[4(4-Chloro-3-{[(5Z)-4-oxo-5-(6-quinolinylmethylidene)-4,5-dihydro-1,3-thiazol-2-yl]amino}phenyl)methyl]glycinamide;
Ethyl (4-chloro-3-{[(5Z)-4-oxo-5-(6-quinolinylmethylidene)-4,5-dihydro-1,3-thiazol-2-yl]amino}phenyl)carbamate;
N-(4-Chloro-3-{[(5Z)-4-oxo-5-(6-quinolinylmethylidene)-4,5-dihydro-1,3-thiazol-2-yl]amino}phenyl)-2-cyclopropylacetamide;
N-(4-Chloro-3-{[(5Z)-4-oxo-5-(6-quinolinylmethylidene)-4,5-dihydro-1,3-thiazol-2-yl]amino}phenyl)-N′-(phenylmethyl)urea;
Ethyl 4-[(4-chloro-3-{[(5Z)-4-oxo-5-(6-quinolinylmethylidene)-4,5-dihydro-1,3-thiazol-2-yl]amino}phenyl)amino]-4-oxobutanoate;
N-(4-Chloro-3-{[(5Z)-4-oxo-5-(6-quinolinylmethylidene)-4,5-dihydro-1,3-thiazol-2-yl]amino}phenyl)-N′-(1-methylethyl)urea;
N-(4-Chloro-3-{[(5Z)-4-oxo-5-(6-quinolinylmethylidene)-4,5-dihydro-1,3-thiazol-2-yl]amino}phenyl)-1-piperidinecarboxamide;
(5Z)-2-{[2-Chloro-5-(1H-imidazol-4-yl)phenyl]amino}-5-(6-quinolinylmethylidene)-1,3-thiazol-4(5H)-one;
(5Z)-2-{[2-Chloro-5-(2-methyl-1,3-thiazol-4-yl)phenyl]amino}-5-(6-quinolinylmethylidene)-1,3-thiazol-4(5H)-one;
5-Chloro-6-{[(5Z)-4-oxo-5-(6-quinolinylmethylidene)-4,5-dihydro-1,3-thiazol-2-yl]amino}-1,3-dihydro-2H-benzimidazol-2-one;
Methyl [(4-chloro-3-{[(5Z)-4-oxo-5-(6-quinolinylmethylidene)-4,5-dihydro-1,3-thiazol-2-yl]amino}phenyl)methyl]carbamate;
N-[(4-Chloro-3-{[(5Z)-4-oxo-5-(6-quinolinylmethylidene)-4,5-dihydro-1,3-thiazol-2-yl]amino}phenyl)methyl]-N′-(1-methylethyl)urea;
2-[3,4-Bis(methyloxy)phenyl]-N-(4-chloro-3-{[(5Z)-4-oxo-5-(6-quinolinylmethylidene)-4,5-dihydro-1,3-thiazol-2-yl]amino}phenyl)acetamide;
(5Z)-2-{[2-Chloro-5-(1,3-oxazol-4-yl)phenyl]amino}-5-(6-quinolinylmethylidene)-1,3-thiazol-4(5H)-one;
(5Z)-2-{[5-(1,3-Benzothiazol-2-yl)-2-chlorophenyl]amino}-5-(6-quinolinylmethylidene)-1,3-thiazol-4(5H)-one;
N-(4-Chloro-3-{[(5Z)-4-oxo-5-(6-quinolinylmethylidene)-4,5-dihydro-1,3-thiazol-2-yl]amino}phenyl)-3-phenylpropanamide;
N-(4-Chloro-3-{[(5Z)-4-oxo-5-(6-quinolinylmethylidene)-4,5-dihydro-1,3-thiazol-2-yl]amino}phenyl)-2-(4-fluorophenyl)acetamide;
(5Z)-2-[(2-Amino-5-chloro-1H-benzimidazol-6-yl)amino]-5-(6-quinolinylmethylidene)-1,3-thiazol-4(5H)-one;
N-(4-Chloro-3-{[(5Z)-4-oxo-5-(6-quinolinylmethylidene)-4,5-dihydro-1,3-thiazol-2-yl]amino}phenyl)-1-phenylmethanesulfonamide;
N′-(4-Chloro-3-{[(5Z)-4-oxo-5-(6-quinolinylmethylidene)-4,5-dihydro-1,3-thiazol-2-yl]amino}phenyl)-N,N-dimethylsulfamide;
(5Z)-2-[(7-Chloro-6-quinoxalinyl)amino]-5-(6-quinolinylmethylidene)-1,3-thiazol-4(5H)-one;
N-(4-{[(4-Chloro-3-{[(5Z)-4-oxo-5-(6-quinolinylmethylidene)-4,5-dihydro-1,3-thiazol-2-yl]amino}phenyl)amino]sulfonyl}-5-methyl-1,3-thiazol-2-yl)acetamide;
N-(4-Chloro-3-{[(5Z)-4-oxo-5-(6-quinolinylmethylidene)-4,5-dihydro-1,3-thiazol-2-yl]amino}phenyl)-2-thiophenesulfonamide;
(5Z)-2-{[2-Chloro-5-(1H-tetrazol-5-yl)phenyl]amino}-5-(6-quinolinylmethylidene)-1,3-thiazol-4(5H)-one;
N-1-(4-Chloro-3-{[(5Z)-4-oxo-5-(6-quinolinylmethylidene)-4,5-dihydro-1,3-thiazol-2-yl]amino}phenyl)glycinamide;
N-(4-Chloro-3-{[(5Z)-4-oxo-5-(6-quinolinylmethylidene)-4,5-dihydro-1,3-thiazol-2-yl]amino}phenyl)-2-(1-pipendinyl)acetamide;
((5Z)-2-{[2-Chloro-5-(2-pyrimidinyl)phenyl]amino}-5-(6-quinolinylmethylidene)-1,3-thiazol-4(5H)-one;
N-(4-Chloro-3-{[(5Z)-4-oxo-5-(6-quinolinylmethylidene)-4,5-dihydro-1,3-thiazol-2-yl]amino}phenyl)-2-cyclopentylacetamide;
N-(4-Chloro-3-{[(5Z)-4-oxo-5-(6-quinolinylmethylidene)-4,5-dihydro-1,3-thiazol-2-yl]amino}phenyl)-2-(1-pyrrolidinyl)acetamide;
N˜1˜-(4-Chloro-3-{[(5Z)-4-oxo-5-(6-quinolinylmethylidene)-4,5-dihydro-1,3-thiazol-2-yl]amino}phenyl)-N˜2˜-ethyl-N˜2˜-methylglycinamide;
N-(4-Chloro-3-{[(5Z)-4-oxo-5-(6-quinolinylmethylidene)-4,5-dihydro-1,3-thiazol-2-yl]amino}phenyl)-2-(4-morpholinyl)acetamide;
1,1-Dimethylethyl 4-{2-[(4-chloro-3-{[(5Z)-4-oxo-5-(6-quinolinylmethylidene)-4,5-dihydro-1,3-thiazol-2-yl]amino}phenyl)amino]-2-oxoethyl}-1-piperazinecarboxylate;
N′-[(4-Chloro-3-{[(5Z)-4-oxo-5-(6-quinolinylmethylidene)-4,5-dihydro-1,3-thiazol-2-yl]amino}phenyl)methyl]-N,N-dimethylimidoformamide;
N-(4-Chloro-3-{[(5Z)-4-oxo-5-(6-quinolinylmethylidene)-4,5-dihydro-1,3-thiazol-2-yl]amino}phenyl)-2-(1-piperazinyl)acetamide;
N-(4-Chloro-3-{[(5Z)-4-oxo-5-(6-quinolinylmethylidene)-4,5-dihydro-1,3-thiazol-2-yl]amino}phenyl)-2-pyridinecarboxamide;
N-(4-Chloro-3-{[(5Z)-4-oxo-5-(6-quinolinylmethylidene)-4,5-dihydro-1,3-thiazol-2-yl]amino}phenyl)cyclohexanecarboxamide;
N-(4-Chloro-3-{[(5Z)-4-oxo-5-(6-quinolinylmethylidene)-4,5-dihydro-1,3-thiazol-2-yl]amino}phenyl)benzamide;
N-(4-Chloro-3-{[(5Z)-4-oxo-5-(6-quinolinylmethylidene)-4,5-dihydro-1,3-thiazol-2-yl]amino}phenyl)cyclopentanecarboxamide;
(5Z)-2-{[2-Chloro-5-(3-thienyl)phenyl]amino}-5-(6-quinolinylmethylidene)-1,3-thiazol-4(5H)-one;
(5Z)-2-({2-Chloro-5-[6-(methyloxy)-2-pyridinyl]phenyl}amino)-5-(6-quinolinylmethylidene)-1,3-thiazol-4(5H)-one;
(5Z)-2-[(3,5-Dichloro-2,6-dimethyl-4-pyridinyl)amino]-5-(6-quinoxalinylmethylidene)-1,3-thiazol-4(5H)-one;
(5Z)-2-{[2-Chloro-5-(4-morpholinyl)phenyl]amino}-5-(6-quinolinylmethylidene)-1,3-thiazol-4(5H)-one;
N˜1˜-(4-Chloro-3-{[(5Z)-4-oxo-5-(6-quinolinylmethylidene)-4,5-dihydro-1,3-thiazol-2-yl]amino}phenyl)leucinamide;
N-(4-Chloro-3-{[(5Z)-4-oxo-5-(6-quinolinylmethylidene)-4,5-dihydro-1,3-thiazol-2-yl]amino}phenyl)-2-methylpropanamide;
(5Z)-2-{[5-(2-Amino-5-pyrimidinyl)-2-chlorophenyl]amino}-5-(6-quinolinylmethylidene)-1,3-thiazol-4(5H)-one;
(5Z)-2-({2-Chloro-5-[(dimethylamino)methyl]phenyl}amino)-5-(6-quinolinylmethylidene)-1,3-thiazol-4(5H)-one;
N-[(4-Chloro-3-{[(5Z)-4-oxo-5-(6-quinolinylmethylidene)-4,5-dihydro-1,3-thiazol-2-yl]amino}phenyl)methyl]methanesulfonamide;
4-Chloro-N,N-dimethyl-3-{[(5Z)-4-oxo-5-(6-quinolinylmethylidene)-4,5-dihydro-1,3-thiazol-2-yl]amino}benzenesulfonamide;
N-(2,4-Dichloro-5-{[(5Z)-4-oxo-5-(6-quinolinylmethylidene)-4,5-dihydro-1,3-thiazol-2-yl]amino}phenyl)cyclobutanecarboxamide;
(5Z)-2-{[4-Chloro-3′-(methyloxy)-3-biphenylyl]amino}-5-(6-quinolinylmethylidene)-1,3-thiazol-4(5H)-one;
(5Z)-2-({2-Chloro-5-[(cyclopentylamino)methyl]phenyl}amino)-5-(6-quinolinylmethylidene)-1,3-thiazol-4(5H)-one;
N-(4-Chloro-3-{[(5Z)-4-oxo-5-(6-quinolinylmethylidene)-4,5-dihydro-1,3-thiazol-2-yl]amino}phenyl)-N-methylcyclobutanecarboxamide;
N-(4-Chloro-3-{[(5Z)-4-oxo-5-(6-quinolinylmethylidene)-4,5-dihydro-1,3-thiazol-2-yl]amino}phenyl)-N-(phenylsulfonyl)benzenesulfonamide;
(5Z)-2-({2-Chloro-5-[(phenylmethyl)amino]phenyl}amino)-5-(6-quinolinylmethylidene)-1,3-thiazol-4(5H)-one;
(5Z)-2-({2-Chloro-5-[(1-methylethyl)amino]phenyl}amino)-5-(6-quinolinylmethylidene)-1,3-thiazol-4(5H)-one;
1,1-Dimethylethyl (4-chloro-3-{[(5Z)-4-oxo-5-(6-quinolinylmethylidene)-4,5-dihydro-1,3-thiazol-2-yl]amino}phenyl)carbamate;
N-(4-Chloro-3-{[(5Z)-4-oxo-5-(6-quinolinylmethylidene)-4,5-dihydro-1,3-thiazol-2-yl]amino}phenyl)-L-prolinamide;
N-1-(4-Chloro-3-{[(5Z)-4-oxo-5-(6-quinolinylmethylidene)-4,5-dihydro-1,3-thiazol-2-yl]amino}phenyl)-L-alaninamide;
1,1-Dimethylethyl 4-{[(4-chloro-3-{[(5Z)-4-oxo-5-(6-quinolinylmethylidene)-4,5-dihydro-1,3-thiazol-2-yl]amino}phenyl)amino]carbonyl}-1-pipendinecarboxylate;
(5Z)-2-({2-Chloro-5-[6-(methylamino)-2-pyridinyl]phenyl}amino)-5-(6-quinolinylmethylidene)-1,3-thiazol-4(5H)-one;
(5Z)-2-[(3,5-Dichloro-4-pyridinyl)amino]-5-(6-quinolinylmethylidene)-1,3-thiazol-4(5H)-one;
1,1-Dimethylethyl (3-chloro-4-{[(5Z)-4-oxo-5-(6-quinolinylmethylidene)-4,5-dihydro-1,3-thiazol-2-yl]amino}phenyl)carbamate;
N-(4-Chloro-3-{[(5Z)-4-oxo-5-(6-quinolinylmethylidene)-4,5-dihydro-1,3-thiazol-2-yl]amino}phenyl)-4-pipendinecarboxamide;
N-(4-Chloro-3-{[(5Z)-4-oxo-5-(6-quinolinylmethylidene)-4,5-dihydro-1,3-thiazol-2-yl]amino}phenypethanesulfonamide;
N-(4-Chloro-3-{[(5Z)-4-oxo-5-(6-quinolinylmethylidene)-4,5-dihydro-1,3-thiazol-2-yl]amino}phenyl)benzenesulfonamide;
N-(4-Chloro-3-{[(5Z)-4-oxo-5-(6-quinolinylmethylidene)-4,5-dihydro-1,3-thiazol-2-yl]amino}phenyl)-1-propanesulfonamide;
N-(3-Chloro-4-{[(5Z)-4-oxo-5-(6-quinolinylmethylidene)-4,5-dihydro-1,3-thiazol-2-yl]amino}phenyl)acetamide;
N-(3-Chloro-4-{[(5Z)-4-oxo-5-(6-quinolinylmethylidene)-4,5-dihydro-1,3-thiazol-2-yl]amino}phenyl)methanesulfonamide;
N-(3-Chloro-4-{[(5Z)-4-oxo-5-(6-quinolinylmethylidene)-4,5-dihydro-1,3-thiazol-2-yl]amino}phenyl)benzamide;
N-(3-Chloro-4-{[(5Z)-4-oxo-5-(6-quinolinylmethylidene)-4,5-dihydro-1,3-thiazol-2-yl]amino}phenyl)-2-methylpropanamide;
N-(4-Chloro-3-{[(5Z)-4-oxo-5-(6-quinolinylmethylidene)-4,5-dihydro-1,3-thiazol-2-yl]amino}phenyl)-4-methylbenzenesulfonamide;
N-(3-Chloro-4-{[(5Z)-4-oxo-5-(6-quinolinylmethylidene)-4,5-dihydro-1,3-thiazol-2-yl]amino}phenyl)-2,2,2-trifluoroacetamide;
(3S)-3-Amino-N-(4-chloro-3-{[(5Z)-4-oxo-5-(6-quinolinylmethylidene)-4,5-dihydro-1,3-thiazol-2-yl]amino}phenyl)butanamide;
N-(4-Chloro-3-{[(5Z)-4-oxo-5-(6-quinolinylmethylidene)-4,5-dihydro-1,3-thiazol-2-yl]amino}phenyl)-L-phenylalaninamide;
N-(4-Chloro-3-{[(5Z)-4-oxo-5-(6-quinolinylmethylidene)-4,5-dihydro-1,3-thiazol-2-yl]amino}phenyl)-4-nitrobenzenesulfonamide;
N-(4-Chloro-3-{[(5Z)-4-oxo-5-(6-quinolinylmethylidene)-4,5-dihydro-1,3-thiazol-2-yl]amino}phenyl)-2,2-dimethylpropanamide;
4-Chloro-N-(2-methylpropyl)-3-{[(5Z)-4-oxo-5-(6-quinoxalinylmethylidene)-4,5-dihydro-1,3-thiazol-2-yl]amino}benzamide;
N-(4-Chloro-3-{[(5Z)-4-oxo-5-(6-quinoxalinylmethylidene)-4,5-dihydro-1,3-thiazol-2-yl]amino}phenyl)propanamide;
N-(4-Chloro-3-{[(5Z)-4-oxo-5-(6-quinoxalinylmethylidene)-4,5-dihydro-1,3-thiazol-2-yl]amino}phenyl)-2-(methyloxy)acetamide;
N-(4-Chloro-3-{[(5Z)-4-oxo-5-(6-quinoxalinylmethylidene)-4,5-dihydro-1,3-thiazol-2-yl]amino}phenyl)-2-(2-thienyl)acetamide;
N-(4-Chloro-3-{[(5Z)-4-oxo-5-(6-quinoxalinylmethylidene)-4,5-dihydro-1,3-thiazol-2-yl]amino}phenyl)-2-methylpropanamide;
N-(4-Chloro-3-{[(5Z)-4-oxo-5-(6-quinoxalinylmethylidene)-4,5-dihydro-1,3-thiazol-2-yl]amino}phenyl)cyclobutanecarboxamide;
N-(4-Chloro-3-{[(5Z)-4-oxo-5-(6-quinoxalinylmethylidene)-4,5-dihydro-1,3-thiazol-2-yl]amino}phenyl)cyclopentanecarboxamide;
(5Z)-2-[(3,5-Dichloro-4-pyridinyl)amino]-5-(6-quinoxalinylmethylidene)-1,3-thiazol-4(5H)-one;
N-(4-Chloro-3-{[(5Z)-4-oxo-5-(6-quinoxalinylmethylidene)-4,5-dihydro-1,3-thiazol-2-yl]amino}phenyl)-4-pipendinecarboxamide;
N-(2-Chloro-3-{[(5Z)-4-oxo-5-(6-quinoxalinylmethylidene)-4,5-dihydro-1,3-thiazol-2-yl]amino}phenyl)-2-methylpropanamide;
N-(4-Chloro-3-{[(5Z)-4-oxo-5-(6-quinoxalinylmethylidene)-4,5-dihydro-1,3-thiazol-2-yl]amino}phenyl)-2,2-dimethylpropanamide;
1,1-Dimethylethyl {2-[(4-chloro-3-{[(5Z)-4-oxo-5-(6-quinoxalinylmethylidene)-4,5-dihydro-1,3-thiazol-2-yl]amino}phenyl)amino]-1,1-dimethyl-2-oxoethyl}carbamate;
N-(2-Chloro-3-{[(5Z)-4-oxo-5-(6-quinoxalinylmethylidene)-4,5-dihydro-1,3-thiazol-2-yl]amino}phenyl)cyclopropanecarboxamide;
N-(2-Chloro-3-{[(5Z)-4-oxo-5-(6-quinoxalinylmethylidene)-4,5-dihydro-1,3-thiazol-2-yl]amino}phenyl)cyclobutanecarboxamide;
N˜1˜-(4-Chloro-3-{[(5Z)-4-oxo-5-(6-quinoxalinylmethylidene)-4,5-dihydro-1,3-thiazol-2-yl]amino}phenyl)-2-methylalaninamide;
N-(4-Chloro-3-{[(5Z)-4-oxo-5-(6-quinoxalinylmethylidene)-4,5-dihydro-1,3-thiazol-2-yl]amino}phenypethanesulfonamide;
N-(4-Chloro-3-{[(5Z)-4-oxo-5-(6-quinoxalinylmethylidene)-4,5-dihydro-1,3-thiazol-2-yl]amino}phenyl)-2-propanesulfonamide;
N-(4-Chloro-3-{[(5Z)-4-oxo-5-(6-quinolinylmethylidene)-4,5-dihydro-1,3-thiazol-2-yl]amino}phenyl)-N-(1-methylethyl)acetamide;
N-(4-Chloro-3-{[(5Z)-4-oxo-5-(6-quinoxalinylmethylidene)-4,5-dihydro-1,3-thiazol-2-yl]amino}phenyl)-N-(1-methylethyl)acetamide;
N-(4-Chloro-3-{[(5Z)-4-oxo-5-(6-quinolinylmethylidene)-4,5-dihydro-1,3-thiazol-2-yl]amino}phenyl)-N-(1-methylethyl)methanesulfonamide;
N-(4-Chloro-3-{[(5Z)-4-oxo-5-(6-quinoxalinylmethylidene)-4,5-dihydro-1,3-thiazol-2-yl]amino}phenyl)-N-(1-methylethyl)methanesulfonamide;
(5Z)-2-({2-Chloro-5-[(1-methylethyl)amino]phenyl}amino)-5-(6-quinoxalinylmethylidene)-1,3-thiazol-4(5H)-one;
N-(2-Chloro-3-{[(5Z)-4-oxo-5-(6-quinoxalinylmethylidene)-4,5-dihydro-1,3-thiazol-2-yl]amino}phenyl)methanesulfonamide;
N-(2-Chloro-3-{[(5Z)-4-oxo-5-(6-quinoxalinylmethylidene)-4,5-dihydro-1,3-thiazol-2-yl]amino}phenyl)benzamide;
1,1-Dimethylethyl {2-[(4-chloro-3-{[(5Z)-4-oxo-5-(6-quinolinylmethylidene)-4,5-dihydro-1,3-thiazol-2-yl]amino}phenyl)amino]-1,1-dimethyl-2-oxoethyl}carbamate;
N¹-(4-Chloro-3-{[(5Z)-4-oxo-5-(6-quinolinylmethylidene)-4,5-dihydro-1,3-thiazol-2-yl]amino}phenyl)-2-methylalaninamide;
N-(4-Chloro-3-{[(5Z)-4-oxo-5-(6-quinolinylmethylidene)-4,5-dihydro-1,3-thiazol-2-yl]amino}phenyl)-2-propanesulfonamide;
4-Chloro-N-[2-(methyloxy)ethyl]-3-{[(5Z)-4-oxo-5-(6-quinolinylmethylidene)-4,5-dihydro-1,3-thiazol-2-yl]amino}benzamide;
4-Chloro-3-{[(5Z)-4-oxo-5-(6-quinolinylmethylidene)-4,5-dihydro-1,3-thiazol-2-yl]amino}-N-propylbenzamide;
4-Chloro-N-(2-hydroxyethyl)-3-{[(5Z)-4-oxo-5-(6-quinolinylmethylidene)-4,5-dihydro-1,3-thiazol-2-yl]amino}benzamide;
4-Chloro-N-[3-(2-oxo-1-pyrrolidinyl)propyl]-3-{[(5Z)-4-oxo-5-(6-quinolinylmethylidene)-4,5-dihydro-1,3-thiazol-2-yl]amino}benzamide;
4-Chloro-N-[2-(4-morpholinyl)ethyl]-3-{[(5Z)-4-oxo-5-(6-quinolinylmethylidene)-4,5-dihydro-1,3-thiazol-2-yl]amino}benzamide;
4-chloro-N-[2-(4-morpholinyl)ethyl]-3-[(4-oxo-4,5-dihydro-1,3-thiazol-2-yl)amino]benzamide;
4-Chloro-N-[3-(4-morpholinyl)propyl]-3-{[(5Z)-4-oxo-5-(6-quinolinylmethylidene)-4,5-dihydro-1,3-thiazol-2-yl]amino}benzamide;
4-Chloro-N-(2-hydroxy-1,1-dimethylethyl)-3-{[(5Z)-4-oxo-5-(6-quinolinylmethylidene)-4,5-dihydro-1,3-thiazol-2-yl]amino}benzamide;
4-Chloro-N-(1-methyl-4-pipendinyl)-3-{[(5Z)-4-oxo-5-(6-quinolinylmethylidene)-4,5-dihydro-1,3-thiazol-2-yl]amino}benzamide;
(5Z)-2-[(2-Chloro-5-{[(3S)-3-hydroxy-1-pyrrolidinyl]carbonyl}phenyl)amino]-5-(6-quinolinylmethylidene)-1,3-thiazol-4(5H)-one;
4-Chloro-3-{[(5Z)-4-oxo-5-(6-quinolinylmethylidene)-4,5-dihydro-1,3-thiazol-2-yl]amino}-N42-(2-thienyl)ethyl]benzamide;
(5Z)-2-{[2-Chloro-5-({-[2-oxo-2-(1-pyrrolidinyl)ethyl]-1-piperazinyl}carbonyl)phenyl]amino}-5-(6-quinolinylmethylidene)-1,3-thiazol-4(5H)-one;
4-Chloro-N-(cyclopropylmethyl)-3-{[(5Z)-4-oxo-5-(6-quinolinylmethylidene)-4,5-dihydro-1,3-thiazol-2-yl]amino}benzamide;
4-Chloro-N-(trans-4-hydroxycyclohexyl)-3-{[(5Z)-4-oxo-5-(6-quinolinylmethylidene)-4,5-dihydro-1,3-thiazol-2-yl]amino}benzamide;
4-Chloro-N-(3-hydroxypropyl)-3-{[(5Z)-4-oxo-5-(6-quinolinylmethylidene)-4,5-dihydro-1,3-thiazol-2-yl]amino}benzamide;
4-Chloro-3-{[(5Z)-4-oxo-5-(6-quinolinylmethylidene)-4,5-dihydro-1,3-thiazol-2-yl]amino}-N-[4-(1-pyrrolidinyl)ethyl]benzamide;
4-Chloro-3-{[(5Z)-4-oxo-5-(6-quinolinylmethylidene)-4,5-dihydro-1,3-thiazol-2-yl]amino}-l -[2-(1-piperidinyl)ethyl]benzamide;
4-Chloro-N-[3-(4-methyl-1-piperazinyl)propyl]-3-{[(5Z)-4-oxo-5-(6-quinolinylmethylidene)-4,5-dihydro-1,3-thiazol-2-yl]amino}benzamide;
(5Z)-2-({2-Chloro-5-[(4-methyl-1-piperazinyl)carbonyl]phenyl}amino)-5-(6-quinolinylmethylidene)-1,3-thiazol-4(5H)-one;
4-Chloro-N-ethyl-3-{[(5Z)-4-oxo-5-(6-quinolinylmethylidene)-4,5-dihydro-1,3-thiazol-2-yl]amino}benzamide;
4-Chloro-N-[2-(dimethylamino)ethyl]-N-methyl-3-{[(5Z)-4-oxo-5-(6-quinolinylmethylidene)-4,5-dihydro-1,3-thiazol-2-yl]amino}benzamide; and
(5Z)-2-{[2-Chloro-5-(4-morpholinylcarbonyl)phenyl]amino}-5-(6-quinolinylmethylidene)-1,3-thiazol-4(5H)-one,
   and/or a pharmaceutically acceptable salt, hydrate, solvate or pro-drug thereof.

When referring to compounds of Formula (IIIAA), the term “aryl” means a cyclic or polycyclic aromatic ring containing from 1 to 14 carbon atoms and optionally containing from one to five heteroatoms, provided that when the number of carbon atoms is 1 the aromatic ring contains at least four heteroatoms, when the number of carbon atoms is 2 the aromatic ring contains at least three heteroatoms, when the number of carbons is 3 the aromatic ring contains at least two heteroatoms and when the number of carbon atoms is 4 the aromatic ring contains at least one heteroatom.

When referring to compounds of Formula (IIIAA), the term “C₁-C₁₂aryl” means a group selected from: phenyl, naphthalene, 3,4-methylenedioxyphenyl, pyridine, biphenyl, quinoline, pyrimidine, quinazoline, thiophene, thiazole, furan, pyrrole, pyrazole, imidazole, indole, oxazole, quinoxaline, 1,3-benzothiazole, indene, pyrazine, 1,3-dihydro-2H-benzimidazole, benzimidazole, benzothiophene, tetrahydrobenzothiophene and tetrazole.

When referring to compounds of Formula (IIIAA), “C₁-C₁₂aryl” is suitably selected from: phenyl, naphthalene, 3,4-methylenedioxyphenyl, pyridine, biphenyl, quinoline, pyrimidine, quinazoline, thiophene, thiazole, furan, pyrrole, pyrazole, imidazole, indole, indene, pyrazine, 1,3-dihydro-2H-benzimidazole, benzimidazole, benzothiophene, tetrahydrobenzothiophene and tetrazole.

When referring to compounds of Formula (IIIAA), the term “substituted” means that the subject chemical moiety has one or more substituents selected from the group consisting of:

aryl,
aryl substituted with one or more subsititents selected from alkyl, hydroxy, alkoxy, amino, alkylamino, alkylamino substituted by oxo, dialkylamino, dialkylamino substituted by one or more oxo groups, oxo, C₁-C₁₂aryl optionally substituted with one or more substituents selected from hydroxy, alkoxy oxo, cyano, amino, alkylamino, dialkylamino, alkyl and alkoxy, cyano, trifluoromethyl, —SO₂NR²¹R²², N-acylamino, —CO₂R²⁰, and halogen,
cycloalkyl substituted with one or more subsititents selected from alkyl, hydroxy, alkoxy, trifluoromethyl, —SO₂NR²¹R²², amino, —CO₂R²⁰, N-acylamino and halogen,
cycloalkyl containing from 1 to 4 heteroatoms substituted with one or more subsititents selected from alkyl, hydroxy, alkoxy, —SO₂NR²¹R²², amino, —CO₂R²⁰, trifluoromethyl, N-acylamino and halogen,
alkoxy substituted with one or more substituents selected form alkyl, —CO₂H, hydroxy, C₁-C₁₂aryl, alkoxy, amino and halogen,
cycloalkyl, cycloalkyl containing from 1 to 4 heteroatoms, C₁-C₄alkylcycloalkyl containing from 1 to 3 heteroatomsC₁-C₄alkyl, —C(O)NHS(O)₂R²⁰, —(CH₂)_{g}NR²³S(O)₂R²⁰, hydroxyalkyl, alkoxy, —(CH₂)_{g}NR²¹R²², —S(O)₂NR²¹R²², —(CH₂)_{g}N(R²⁰)C(O)ₘR²⁰, —(CH₂)_{g}N═C(H)R⁵⁰ where R⁵⁰ is selected from amine, alkylamine and dialkylamine, —(CH₂)_{g}C(O)ₘR²⁰, acyloxy, alkyl, —OCF₃, amino, hydroxy, alkylamino, acetamide, aminoalkyl, aminoalkoxy, alkylaminoalkoxy, dialkylaminoalkoxy, alkoxyalkylamide, alkoxyC₁-C₁₂aryl, C₁-C₁₂aryl, C₁-C₁₂arylalkyl, dialkylamino, N-acylamino, aminoalkylN-acylamino, —(CH₂)_{g}S(O)ₙR²³, nitro, cyano, oxo, halogen, trifluoromethyloxy and trifluoromethyl;
where
g is 0 to 6,
n is 0 to 2,
m is 1 or 2;
R²³ is selected from hydrogen, C₁-C₁₂aryl, C₁-C₁₂aryl substituted with one or more substituents selected from C₁-C₆alkyl and halogen, alkylamino substituted by oxo, dialkylamino substituted by one or more oxo groups, C₁-C₁₂arylC₁-C₆alkyl, C₁-C₁₂arylC₁-C₆alkyl substituted with one or more substituents selected from C₁-C₆alkyl and halogen, or alkyl,
each R²⁰ is independently selected form hydrogen, hydroxy, alkyl optionally substituted with one or more substituents selected from hydroxy and halogen, C₁-C₆alkyloxyC₁-C₆alkyl, C₁-C₆alkyloxyC₁-C₆alkylamine, C₁-C₄alkylC(O)OC₁-C₄alkyl, —C₁-C₆alkylC(O)OH, amino, alkylamino where the alkyl is optionally substituted with one or more substituents selected from hydroxy, oxo, cycloalkyl containing from 1 to 4 heteroatoms where cycloalkyl containing from 1 to 4 heteroatoms is optionally substituted by one or more substituents selected form C₁-C₆alkyl, halogen and oxo, and C₁-C₆alkyl, dialkylamino where each alkyl is independently and optionally substituted with one or more substituents selected from hydroxyl, oxo, C₁-C₆alkyl, amino, alkylamino and dialkylamino, aminoC₁-C₆alkyl, alkylaminoC₁-C₆alkyl where the alkyl is optionally substituted by one or more substituents selected from oxo, alkoxy and halogen, dialkylaminoC₁-C₆alkyl where each alkyl is independently and optionally substituted with one or more substituents selected from hydroxy, oxo, C₁-C₆alkyl, amino, alkylamino and dialkylamino, —C(O)OH, alkoxy, aryloxy, arylamino, diarylamino, arylalkylamino where the aryl is optionally substituted with one or more substituents selected from hydroxyl, alkoxy, hydroxyalkyl, oxo, C₁-C₆alkyl, amino, alkylamino and dialkylamino, cycloalkylalkylamino, aryl, aryl substituted with one or more substituents selected from oxo, hydroxyl, —N(H)C(O)C₁-C₆alkyl, alkoxy, nitro, amine and alkyl, arylC₁-C₄alkyl optionally substituted with one or more substituents selected from oxo, amino, alkylamino, alkylamino substituted by oxo, dialkylamino, dialkylamino substituted by one or more oxo groups, hydroxy, halogen, alkoxy and alkyl, —CH₂C(O)cycloalkyl containing from 1 to 4 heteroatoms, cycloalkylC₁-C₆alkyl where the cycloalkyl is optionally substituted with one or more substituents selected from C₁-C₆alkyl, oxo, halogen, —C(O)OC₁-C₆alkyl and —C₁-C₆alkylC(O)OH, C₁-C₄alkyl substituted with cycloalkyl containing from 1 to 4 heteroatoms where the cycloalkyl containing from 1 to 4 heteroatoms is optionally substituted with one or more substituents selected from C₁-C₆alkyl, oxo, halogen, —C(O)OC₁-C₆alkyl and —C₁-C₆alkylC(O)OH, cycloalkyl, —N(H)cycloalkyl, cycloalkyl substituted with one or more substituents selected from oxo, hydroxy, halogen, amino, alkylamino, dialkylamino, —C(O)OH, —C(O)NR⁸⁰R⁹⁰ where R⁸⁰ and R⁹⁰ are each independently selected form hydrogen and C₁-C₈alkyl, and alkyl, —N(H)cycloalkyl substituted with one or more substituents selected from oxo, hydroxy and alkyl, cycloalkyl containing from 1 to 4 heteroatoms, cycloalkyl containing from 1 to 4 heteroatoms substituted with one or more substituents selected from oxo, alkoxy, hydroxyl and alkyl where alkyl is optionally substituted with one or more substituents selected from halogen, hydroxy, alkoxy, oxo and cycloalkyl containing from 1 to 4 heteroatoms, —N(H)cycloalkyl containing from 1 to 4 heteroatoms substituted with one or more substituents selected from oxo, hydroxy and alkyl, and trifluoromethyl, and
R²¹ and R²² are independently selected form hydrogen, alkyl, C₁-C₆alkyl substituted with one of more substituents selected from hydroxy, cycloalkyl containing from 1 to 4 heteroatoms optionally substituted with one or more substituents selected from C₁-C₆alkyl, hydroxy, oxo and halogen, ═NH, and EN, —S(O)₂aryl where aryl is optionally substituted with one or more substituents selected from: halogen, alkylamino and dialkylamino, C₁-C₁₂aryl, cycloalkyl containing from 1 to 4 heteroatoms, cycloalkyl containing from 1 to 4 heteroatoms substituted with one or more substituents selected from oxo, hydroxy, and alkyl, cycloalkyl, cycloalkyl substituted with one or more substituents selected from oxo, hydroxy, and alkyl, arylC₁-C₆alkyl optionally substituted with one or more substituents selected from oxo, hydroxy, and alkyl, cycloalkyl containing from 1 to 4 heteroatoms optionally substituted with one or more substituents selected from oxo, hydroxyl and alkyl, C₁-C₆alkoxy, C₁-C₄alkyloxyC₁-C₄alkyl, aryl and trifluoromethyl.

When referring to compounds of Formula (IIIAA), the term “substituted” suitably means that the subject chemical moiety has from one to five of the indicated substituents, suitably, from one to four of the indicated substituents, suitably from one to three of the indicated substituents, suitably one or two of the indicated substituents.

When referring to compounds of Formula (IIIAA), the term “alkoxy” means —Oalkyl where alkyl is as described for Formula (IIIAA) including —OCH₃ and —OC(CH₃)₂CH₃.

When referring to compounds of Formula (IIIAA), term “cycloalkyl” means a nonaromatic, unsaturated or saturated, cyclic or polycyclic C₃-C₁₂.

When referring to compounds of Formula (IIIAA), examples of cycloalkyl and substituted cycloalkyl substituents include: cyclohexyl, aminocyclohexyl, cyclobutyl, aminocyclobutyl, 4-hydroxy-cyclohexyl, 2-ethylcyclohexyl, propyl4-methoxycyclohexyl, 4-methoxycyclohexyl, 4-carboxycyclohexyl, cyclopropyl, aminocyclopentyl, and cyclopentyl.

When referring to compounds of Formula (IIIAA), the term “cycloalkyl containing from 1 to 4 heteroatoms” and the term “cycloalkyl containing from 1 to 3 heteroatoms” means a nonaromatic, unsaturated or saturated, cyclic or polycyclic ring containing from 1 to 12 carbons and containing from one to four heteroatoms or from one to three heteroatoms (respectively), provided that when the number of carbon atoms is 1 the aromatic ring contains at least four heteroatoms (applicable only where “cycloalkyl containing from 1 to 4 heteroatoms” is indicated), when the number of carbon atoms is 2 the aromatic ring contains at least three heteroatoms, when the number of carbon atoms is 3 the nonaromatic ring contains at least two heteroatoms and when the number of carbon atoms is 4 the nonaromatic ring contains at least one heteroatom.

When referring to compounds of Formula (IIIAA), examples of cycloalkyl containing from 1 to 4 heteroatoms, cycloalkyl containing from 1 to 3 heteroatoms, substituted cycloalkyl containing from 1 to 4 heteroatoms and substituted cycloalkyl containing from 1 to 3 heteroatoms include: piperidine, piperazine, pyrrolidine, 3-methylaminopyrrolidine, piperazinly, tetrazole, hexahydrodiazepine and morpholine.

When referring to compounds of Formula (IIIAA), the term “acyloxy” means —OC(O)alkyl where alkyl is as described for Formula (IIIAA). Examples of acyloxy substituents include: —OC(O)CH₃, —OC(O)CH(CH₃)₂ and —OC(O)(CH₂)₃CH₃.

When referring to compounds of Formula (IIIAA), the term “N-acylamino” means —N(H)C(O)alkyl, where alkyl is as described for Formula (IIIAA). Examples of N-acylamino substituents include: —N(H)C(O)CH₃, —N(H)C(O)CH(CH₃)₂ and —N(H)C(O)(CH₂)₃CH₃.

When referring to compounds of Formula (IIIAA), the term “aryloxy” means —Oaryl where aryl is phenyl, naphthyl, 3,4-methylenedioxyphenyl, pyridyl or biphenyl optionally substituted with one or more substituents selected from the group consisting of: alkyl, hydroxyalkyl, alkoxy, trifluoromethyl, acyloxy, amino, N-acylamino, hydroxy, —(CH₂)_{g}C(O)OR²⁵, —S(O)ₙR²⁵, nitro, cyano, halogen and protected —OH, where g is 0-6, R²⁵ is hydrogen or alkyl, and n is 0-2. Examples of aryloxy substituents include: phenoxy, 4-fluorophenyloxy and biphenyloxy.

When referring to compounds of Formula (IIIAA), the term “heteroatom” means oxygen, nitrogen or sulfur.

When referring to compounds of Formula (IIIAA), the term “halogen” means a substituent selected from bromide, iodide, chloride and fluoride.

When referring to compounds of Formula (IIIAA), the term “alkyl” and derivatives thereof and in all carbon chains, including alkyl chains defined by the term “—(CH₂)ₙ”, “—(CH₂)ₘ” and the like, means a linear or branched, saturated or unsaturated hydrocarbon chain, and unless otherwise defined, the carbon chain will contain from 1 to 12 carbon atoms. Examples of alkyl and substituted alkyl substituents include: —CH₃, —CH₂—CH₃, —CH₂—CH₂—CH₃, —CH(CH₃)₂, —CH₂—CH₂—C(CH₃)₃, —CH₂—CF₃, —C≡C—C(CH₃)₃, —C≡C—CH₂—OH, cyclopropylmethyl, —CH₂—C(CH₃)₂—CH₂—NH₂, —C≡C—C₆H₅, —C≡C—C(CH₃)₂—OH, —CH₂—CH(OH)—CH(OH)—CH(OH)—CH(OH)—CH₂—OH, piperidinylmethyl, methoxyphenylethyl, —C(CH₃)₃, —(CH₂)₃—CH₃, —CH₂—CH(CH₃)₂, —CH(CH₃)—CH₂—CH₃, —CH═CH₂, and —C≡C—CH₃.

When referring to compounds of Formula (IIIAA), the crisscrossed double bond indicated by the symbol “ [symbol] ” denotes Z and/or E stereochemistry around the double bond. In other words a compound of Formula (IIIAA) can be either in the Z or E stereochemistry around this double bond, or a compound of Formula (IIIAA) can be in a mixture of Z and E stereochemistry around the double bond.

Further definitions and descriptions of the compounds of Formula (IIIAA), including those directed to “tautomers”, are found in International Application No. PCT/US2006/019447, and are incorporated herein by reference.

By the term “selected disease state”, and derivatives thereof, refers to a disease state suitable for treatment according to the current invention. Such disease states include: Alzheimer's disease, Down's syndrome, mental retardation, memory defects, memory loss, pancreatic cancer, bone resorption disease, osteoporosis, sickle cell anemia, chronic kidney disease, diabetes, depression, and subsets of depression including: alcoholism, anxiety, obsessive compulsive disorder, panic disorder, chronic pain, obesity, senile dementia, migraine, bulimia, anorexia, social phobia, pre-menstrual syndrome (PMS), adolescent depression, trichotillomania, dysthymia and substance abuse. Also included in the methods of the present invention is the enhancement of cognition.

By the term “treating” and derivatives thereof as used herein, is meant prophylactic and therapeutic therapy. Prophylactic therapy for cancer is appropriate, for example, when a subject is considered at high risk for developing cancer (such as an individual with a strong family history of cancer), or when an individual has been exposed to a carcinogen.

By the phrases “to a therapeutic extent”, “treating” and “therapeutically effective amount” and derivatives thereof as used herein, unless otherwise defined, is meant that amount of the selected substituted thiazol compound that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, lessening in severity or amelioration of a disease or disorder. The term also includes within its scope amounts effective to enhance normal physiological function.

The selected substituted thiazol compounds of the invention are used in the methods of the invention. Where a —COOH or —OH group is present, pharmaceutically acceptable esters can be employed, for example methyl, ethyl, pivaloyloxymethyl, and the like for —COOH, and acetate maleate and the like for —OH, and those esters known in the art for modifying solubility or hydrolysis characteristics, for use as sustained release or prodrug formulations.

By the term “co-administering” and derivatives thereof as used herein is meant either simultaneous administration or any manner of separate sequential administration of a selected substituted thiazol compound, as described herein, and a further active ingredient or ingredients, known to be useful in the treatment of a selected disease state, as described herein, including chemotherapy and radiation treatment when the disease state is pancreatic cancer. The term further active ingredient or ingredients, as used herein, includes any compound or therapeutic agent known to or that demonstrates advantageous properties when administered to a patient in need of treatment for a selected disease state. Preferably, if the administration is not simultaneous, the compounds are administered in a close time proximity to each other. Furthermore, it does not matter if the compounds are administered in the same dosage form, e.g. one compound may be administered topically and another compound may be administered orally.

The current invention relates to the use of selected substituted thiazol compounds in the treatment of selected disease states in mammals, including humans.

Prophylactic use of the compounds of this invention is contemplated whenever numerous causative factors are present in a subject. For example prophylactic use for the treatment of pancreatic cancer includes but is not limited to treatment of heavy coffee drinkers with no detectable cancer.

The present invention therefore provides a method of treating one or more disease states selected from: Alzheimer's disease, pancreatic cancer bone resorption disease, osteoporosis, sickle cell anemia, chronic kidney disease, Down's syndrome, mental retardation, memory defects, memory loss, diabetes, depression, and subsets of depression including: alcoholism, anxiety, obsessive compulsive disorder, panic disorder, chronic pain, obesity, senile dementia, migraine, bulimia, anorexia, social phobia, pre-menstrual syndrome (PMS), adolescent depression, trichotillomania, dysthymia and substance abuse which comprises the administration of a therapeutically effective amount of a selected substituted thiazol compound.

Further, the selected substituted thiazol compounds of the invention have utility as cognition enhancers. Thus, the present invention provides a method of enhancing cognition which comprises the administration of a therapeutically effective amount of a selected substituted thiazol compound as disclosed herein.

The drug may be administered to a patient in need thereof by any conventional route of administration, including, but not limited to, intravenous, intramuscular, oral, subcutaneous, intradermal, and parenteral.

The selected substituted thiazol compound of the present invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The pharmaceutical preparations are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the pharmaceutically active compounds in a pharmaceutical dosage unit as described above will be an efficacious, nontoxic quantity preferably selected from the range of 0.001-100 mg/kg of active compound, preferably 0.002-50 mg/kg. When treating a human patient in need of a selected substituted thiazol compound, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg, more preferably 0.1 to 3000 mg of active compound. Oral administration, which uses lower dosages is preferred. Parenteral administration, at high dosages, however, also can be used when safe and convenient for the patient.

Optimal dosages to be administered may be readily determined by those skilled in the art, and will vary with the particular selected substituted thiazol compound in use, the strength of the preparation, the mode of administration, and the advancement of the disease condition. Additional factors depending on the particular patient being treated will result in a need to adjust dosages, including patient age, weight, diet, and time of administration.

The method of this invention of treating a selected disease state in mammals, including humans, comprises administering to a subject in need thereof a therapeutically effective amount of a selected substituted thiazol compound of the present invention.

The present invention relates to the use of a selected substituted thiazol compound in the treatment of a selected disease state in a mammal, including a human.

The present invention relates to the in vivo administration of a selected substituted thiazol compound in the treatment a selected disease state in a mammal, including a human.

The invention also provides for the use of a compound of selected substituted thiazol compound in the manufacture of a medicament for use in the treatment of a selected disease state in mammals including humans.

The invention also provides for the use of a compound of selected substituted thiazol compound in the manufacture of a medicament for use in therapy.

The invention also provides for a pharmaceutical composition for use in the treatment of a selected disease state, which comprises a selected substituted thiazol compound and a pharmaceutically acceptable carrier.

The invention also provides for the use of a compound of a selected substituted thiazol compound in the manufacture of a medicament for use in therapy.

No unacceptable toxicological effects are expected when compounds of the invention are administered in accordance with the present invention.

In addition, the pharmaceutically active compounds of the present invention can be co-administered with further active ingredients, such as other compounds known to treat the a selected disease state, as defined herein.

Contemplated Equivalents—It will be appreciated by the person of ordinary skill in the art that the selected substituted thiazol compounds of the invention may also exist in tautomeric forms. All tautomeric, isomeric and all such forms of the substituted thiazol compounds are included in scope of the current invention.

Without further elaboration, it is believed that one skilled in the art can, using the preceding description, utilize the present invention to its fullest extent. The following Examples are, therefore, to be construed as merely illustrative and not a limitation of the scope of the present invention in any way.

### EXPERIMENTAL DETAILS

### Example 1

### Capsule Composition

An oral dosage form for administering the present invention is produced by filing a standard two piece hard gelatin capsule with the ingredients in the proportions shown in Table I, below.

TABLE I

| INGREDIENTS | AMOUNTS |
|---|---|
| (5Z)-5-(6-quinoxalinylmethylidene)-2-[(2,6- | 25 mg |
| dichlorophenyl)amino]-1,3-thiazol-4(5H)-one | |
| (Disclosed in WO2006/135712) | |
| Lactose | 55 mg |
| Talc | 16 mg |
| Magnesium Stearate | 4 mg |

### Example 2

### Injectable Parenteral Composition

An injectable form for administering the present invention is produced by stirring 1.5% by weight of (5Z)-5-(6-quinoxalinylmethylidene)-2-[(2,6-dichlorophenyl)amino]-1,3-thiazol-4(5H)-one meglumine salt (Disclosed in WO2006/135712) in 10% by volume propylene glycol in water.

### Example 3

### Tablet Composition

The sucrose, calcium sulfate dihydrate and a non-peptide TPO agonist, as shown in Table II below, are mixed and granulated in the proportions shown with a 10% gelatin solution. The wet granules are screened, dried, mixed with the starch, talc and stearic acid, then screened and compressed into a tablet.

TABLE II

| INGREDIENTS | AMOUNTS |
|---|---|
| 5Z)-5-(6-quinoxalinylmethylidene)-2-[(2,6- | 20 mg |
| dichlorophenyl)amino]-1,3-thiazol-4(5H)-one | |
| (Disclosed in WO2006/135712) | |
| calcium sulfate dihydrate | 30 mg |
| sucrose | 4 mg |
| starch | 2 mg |
| talc | 1 mg |
| stearic acid | 0.5 mg |

While the preferred embodiments of the invention are illustrated by the above, it is to be understood that the invention is not limited to the precise instructions herein disclosed and that the right to all modifications coming within the scope of the following claims is reserved.

What is claimed is:

1. The method of treating a disease state selected from; Alzheimer's disease, Down's syndrome, mental retardation, memory defects, memory loss, pancreatic cancer, bone resorption disease, osteoporosis, sickle cell anemia, chronic kidney disease, diabetes, depression, and subsets of depression including: alcoholism, anxiety, obsessive compulsive disorder, panic disorder, chronic pain, obesity, senile dementia, migraine, bulimia, anorexia, social phobia, pre-menstrual syndrome (PMS), adolescent depression, trichotillomania, dysthymia and substance abuse, in a mammal in need thereof which comprises administering to such mammal a therapeutically effective amount of a selected substituted thiazol compound of Formula (IAA): 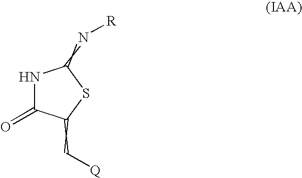
in which
R is C₃₋₆ cycloalkyl or naphtyl; or
R is 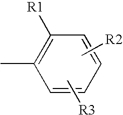
in which R1 is hydrogen, halogen, —C₁₋₆alkyl, —SC₁₋₆alkyl, —NO₂, —S(═O)—C₁₋₆alkyl, —OH, —CF₃, —CN, —CO₂H, —OCF₃, or —CO₂C₁₋₆alkyl;
and R2 and R3 are independently hydrogen, halogen, —C₁₋₆ alkyl, —OC₁₋₆alkyl, —NO₂, —S(═O)—C₁₋₆alkyl, —OH, —CF₃, —CN, —CO₂H, —CO₂C₁₋₆alkyl, —CONH₂, —NH₂, —OCH₂(C═O)OH, —OCH₂CH₂OCH₃, —SO₂NH₂, —CH₂SO₂CH₃, —NH(C═NH)CH₃; or R2 and R3 can independently be a radical of the formula 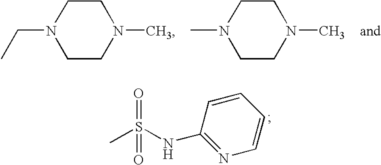
or
R is 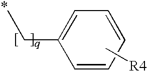
in which q is one or two; R⁴ is hydrogen, halogen, or —SO₂NH₂; or
R is —(CH₂)ₙ—NR^{k}R^{l} in which n is 2 or 3, and R^{k} and R^{l} are independently —C₁₋₆alkyl; or —NR^{k}R^{l} together form 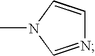
or
R is 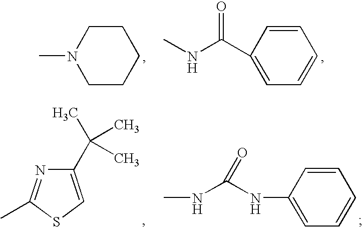
and
Q is 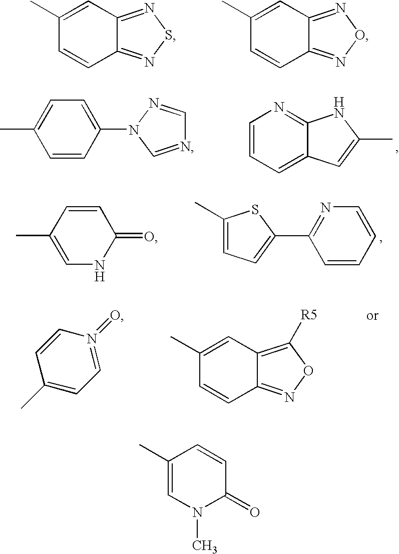
in which R5 is hydrogen, phenyl optionally substituted with up to three C₁₋₆ alkyl or halogen, or C₁₋₆ alkyl; or
Q is 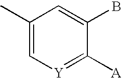
in which Y is CH; and A and B together are a part of 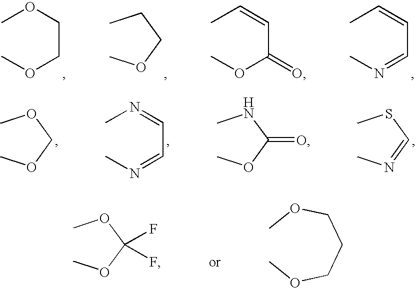
provided that ortho position to Y is N or O; or
Q is 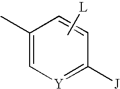
in which Y is N or CH; J is hydrogen, NH₂, OH or —OC₁₋₆alkyl; and L is hydrogen, NH₂, halogen, —NO₂, or —OC₁₋₆alkyl;
or a pharmaceutically acceptable salt thereof.

2. The method of enhancing cognition in a mammal in need thereof which comprises administering to such mammal a therapeutically effective amount of a selected substituted thiazol compound, as described in claim 1.

3. The method of treating a disease state selected from; Alzheimer's disease, Down's syndrome, mental retardation, memory defects, memory loss, pancreatic cancer, bone resorption disease, osteoporosis, diabetes, depression, and subsets of depression including: alcoholism, anxiety, obsessive compulsive disorder, panic disorder, chronic pain, obesity, senile dementia, migraine, bulimia, anorexia, social phobia, pre-menstrual syndrome (PMS), adolescent depression, trichotillomania, dysthymia and substance abuse, in a mammal in need thereof which comprises administering to such mammal a therapeutically effective amount of a selected substituted thiazol compound, as described in claim 1.

4. The method of treating a disease state selected from sickle cell anemia and chronic kidney disease in a mammal in need thereof which comprises administering to such mammal a therapeutically effective amount of a selected substituted thiazol compound, as described in claim 1.

5. A method according to claim 1 wherein the mammal is a human.

6. A method according to claim 1 wherein the compound is (5Z)-5-(6-quinoxalinylmethylidene)-2-[(2,6-dichlorophenyl)amino]-1,3-thiazol-4(5H)-one and/or a pharmaceutically acceptable salt thereof.

7. A method according to claim 1 wherein the compound is (5Z)-5-(6-quinoxalinylmethylidene)-2-[(2,6-dichlorophenyl)amino]-1,3-thiazol-4(5H)-one meglumine.

8. (canceled)

9. (canceled)

10. A method according to claim 6 wherein the mammal is a human.

11. A method according to claim 7 wherein the mammal is a human.

12. (canceled)

13. (canceled)

14. (canceled)

15. The method of treating a subset of depression including: alcoholism, anxiety, obsessive compulsive disorder, panic disorder, chronic pain, obesity, senile dementia, migraine, bulimia, anorexia, social phobia, pre-menstrual syndrome (PMS), adolescent depression, trichotillomania, dysthymia and substance abuse in a mammal in need thereof, which comprises administering to such mammal a therapeutically effective amount of a DYRK1a inhibiting compound.

16. The method of enhancing cognition in a mammal in need thereof, which comprises administering to such mammal a therapeutically effective amount of a DYRK1a inhibiting compound.

17. A method according to claim 1 wherein the compound is (5Z)-5-(6-quinoxalinylmethylidene)-2-[(2,4,6-trichlorophenyl)amino]-1,3-thiazol-4(5H)-one and/or a pharmaceutically acceptable salt thereof.
